(19) United States
(12) Patent Application Publication
(10) Pub. No.: US 2025/0197459 A1
(43) Pub. Date: Jun. 19, 2025

(54) MECHANICALLY TUNABLE, SCALABLE AND COMPOSTABLE AQUAPLASTIC

(71) Applicant: Northeastern University, Boston, MA (US)

(72) Inventors: Avinash Manjula Basavanna, Boston, MA (US); Anna M. Duraj-Thatte, Kensington, CA (US); Neel Satish Joshi, Somerville, MA (US)

(21) Appl. No.: 18/965,622

(22) Filed: Dec. 2, 2024

Related U.S. Application Data
(60) Provisional application No. 63/604,497, filed on Nov. 30, 2023.

(51) Int. Cl. C07K 14/245; C12N 1/20; C12N 15/62

(52) U.S. Cl. CPC C07K 14/245; C12N 1/20; C12N 15/62; C07K 2319/00

(57) ABSTRACT

Provided are engineered living materials including engineered fibers and engineered microbial cells. The engineered fibers include a first Curli protein fused to a first binding peptide and a second Curli protein fused to a second binding peptide, wherein the first binding peptide is covalently linked to the second binding peptide, crosslinking the first Curli protein with the second Curli protein.

### RELATED APPLICATIONS

This application claims the benefit of priority to U.S. Patent Application Ser. No. 63/604,497, filed Nov. 30, 2023; which is incorporated by reference in its entirety.

### GOVERNMENT SUPPORT

This invention was made with government support under Grant No. DMR 2004875 and DMR-1410751 were awarded by the National Science Foundation. The government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Feb. 6, 2025, is named NEX-17201_SL.xml and is 19,047 bytes in size.

### BACKGROUND

With over 8.3 billion tons, plastics are one of the largest human-made materials, and 79% of which have accumulated in landfills and oceans. In addition, the contamination of microplastics in almost all parts of the globe further enhances their threat to our health and the environment. Biodegradable bioplastics account for less than 1% of the global plastic market and their limited properties warrants the development of new alternatives. Given that the typical lifetime of a packaging material is 1-2 years, and the packaging industry accounts for nearly one third of the plastic market, there lies a tremendous scope and opportunity for biodegradable packages. It is to be noted that water soluble polymers like PVA (commonly found in detergent pods) have limited biodegradation under diverse settings of land and water. In many cases, the biodegradable polymers like PVA are blended with petrochemical plastics to enhance certain material properties but limits their water dispersibility and biodegradability. Accordingly, there is need for developing a material that integrates the features of compostability, healability, and scalability.

### SUMMARY OF THE INVENTION

In one aspect the present disclosure provides an engineered fiber, comprising a first Curli protein, and a second Curli protein. In some embodiments, the first Curli protein is fused to a first binding peptide, and the second Curli protein is fused to a second binding peptide. In some embodiments, the first binding peptide is linked to the second binding peptide. In some embodiments, the first binding peptide is covalently linked to the second binding peptide. In some embodiments, the first Curli protein and the second Curli protein are CsgA. In some embodiments, the CsgA comprises the amino acid sequence set forth in SEQ ID NO: 1.

In some embodiments, the first binding peptide and the second binding peptide are any one of the following pairs: SpyCatcher and Spytag, SnoopCatcher and Snooptag, and streptavidin and biotin. In some embodiments, the first binding peptide and the second binding peptide are SpyCatcher and Spytag; and the Spytag comprises the amino acid sequence set forth in SEQ ID NO: 3 and the SpyCatcher comprises the amino acid sequence set forth in SEQ ID NO: 4. In some embodiments, the first Curli protein is fused to the first binding peptide via a spacer and/or linker. In some embodiments, the second Curli protein is fused to the second binding peptide via a linker and/or a spacer. In some embodiments, the linker is glycine serine linker. In some embodiments, the glycine serine liner comprises the amino acid sequence set forth in any one of SEQ ID NOs: 2, 11, and 13-18 and the sequence GSG. In some embodiments, the spacer comprises an intrinsically disordered protein. In some embodiments, the intrinsically disordered protein is TDP-43, FUS, SMN, ataxin-2, optineurin, angiogenin, tau, α-synuclein, huntingtin, DRPLA protein, elastin, or casein. In some embodiments, the casein comprises the amino acid sequence set forth in SEQ ID NO: 5. In some embodiments, the fiber is a nonfiber (e.g., a diameter of 1-100 nanometers).

In some embodiments, the engineered fiber has a Young's modulus of 6-450 MPa. In some embodiments, the engineered fiber has an elongation at break 1-160%.

In another aspect the present disclosure provides an engineered microbial cell comprising an exogenous nucleic acid encoding a first Curli protein and a second Curli protein. In some embodiments, the first Curli protein is fused to a first binding peptide, and the second Curli protein is fused to a second binding peptide. In some embodiments, the first binding peptide is linked to the second binding peptide. In some embodiments, the first binding peptide is covalently linked to the second binding peptide. In some embodiments, the first Curli protein and the second Curli protein are CsgA. In some embodiments, the CsgA comprises the amino acid sequence set forth in SEQ ID NO: 1. In some embodiments, the first binding peptide and the second binding peptide are any one of the following pairs: SpyCatcher and Spytag, SnoopCatcher and Snooptag, and streptavidin and biotin. In some embodiments, the first binding peptide and the second binding peptide are SpyCatcher and Spytag; and the Spytag comprises the amino acid sequence set forth in SEQ ID NO: 3 and the SpyCatcher comprises the amino acid sequence set forth in SEQ ID NO: 4.

In some embodiments, the first Curli protein is fused to the first binding peptide via a spacer and/or linker. In some embodiments, the second Curli protein is fused to the second binding peptide via a linker and/or a spacer. In some embodiments, the linker is glycine serine linker. In some embodiments, the glycine serine liner comprises the amino acid sequence set forth in any one of SEQ ID NOs: 2, 11, and 13-18 and the sequence GSG. In some embodiments, the spacer comprises an intrinsically disordered protein. In some embodiments, the intrinsically disordered protein is TDP-43, FUS, SMN, ataxin-2, optineurin, angiogenin, tau, α-synuclein, huntingtin, DRPLA protein, elastin, or casein. In some embodiments, the casein comprises the amino acid sequence set forth in SEQ ID NO: 5.

In some embodiments, the engineered microbial cell is a bacterial or a fungal cell. In some embodiments, the engineered microbial cell is Escherichia coli, Lactobacillus rhamnosus, Salmonella spp, or Saccharomyces cerevisiae. In some embodiments, the engineered microbial cell is Escherichia coli strain PQN4.

In another aspect the present disclosure provides an engineered living material, comprising a plurality of the engineered microbial cells disclosed herein and the engineered fiber disclosed herein. In some embodiments, the engineered living material comprises 40% cultured bacterial biomass. In some embodiments, the engineered living material is produced in scalable quantities. In some embodiments, the engineered living material biodegrade completely in 15-75 days.

In another aspect the present disclosure provides a method of fabricating an engineered living material. The method may comprise proliferating a plurality of engineered microbial cells disclosed herein, thereby producing the engineered fiber and engineered microbial cells. The method may further comprise isolating the engineered microbial cells and engineered fiber. The method may further comprise allowing the isolated cells and engineered fiber to dry; thereby forming the engineered living material.

In some embodiments, the engineered living material exhibits properties similar to plastic or paper. In some embodiments, the engineered living material is a flexible film. In some embodiments, the method further comprises casting the engineered living material and drying the engineered living material on a silicone mold. In some embodiments, the method further comprises treating the engineered living material with glycerol. In some embodiments, the method further comprises treating the engineered living material with sodium dodecyl sulfate (SDS).

### BRIEF DESCRIPTION OF THE DRAWINGS

FIGS. 1A-1D show schematic summary of mechanically engineered living materials with compostability, healability and scalability (MECHS). Native (FIG. 1A) and functional Curli nanofibers (FIGS. 1B-1C) were separately produced from engineered Escherichia coli.

FIG. 1D shows that the treated biomass of engineered E. coli was dried ambiently to biofabricate MECHS films in a scalable manner. MECHS films exhibit plastic-like stretchability, mechanical tunability and skin-like healability. Parts of the schematics were adapted from BioRender.

FIGS. 2A-2J show mechanical properties of MECHS. FIG. 2A shows genetic design of E. coli to produce Curli nanofibers. FIG. 2B shows representative stress strain curves of MECHS treated with 0 to 5% plasticizer. FIG. 2C shows elongation at break, Young's modulus (FIG. 2D) and Ultimate tensile strength (FIG. 2D) of MECHS treated with 0 to 5% plasticizer. Biological replicates n=10. Data represented as mean±standard deviation. FIG. 2C *p=0.0132, ****p<0.0001. FIG. 2D *p=0.029, **p=0.0021, **p=0.0011, ***p=0.0009, ***p=0.0007. FIG. 2E *p=0.1203, ***p=0.0009, ****p<0.0001. One-way ANOVA followed by Tukey's multiple comparisons test. FIGS. 2F-2J show representative photographs of tensile tests of MECHS films with a lateral dimension of 0.5 cm by 4 cm. 1% (FIG. 2F), 2% (FIG. 2G), 3% (FIG. 2H), 4% (FIG. 2I), and 5% (FIG. 2J) of plasticizer. Left image: initial. Right image: before break.

FIGS. 3A-3I show tailoring the mechanical properties of MECHS through genetic engineering. Genetic design of E. coli to produce the functional Curli nanofibers to covalently crosslink (FIG. 3A) CL1: SpyTag and SpyCatcher (SpyCat) domains fused to CsgA, (FIG. 3B) CL2: SpyTag and SpyCat domains fused to CsgA via the Spacer. FIG. 3C shows representative stress strain curves of MECHS films consisting of CsgA, CL1 and CL2 with 3% plasticizer. Young's modulus (FIG. 3D), and Elongation (FIG. 3E) at break for CsgA, CL1 and CL2 with 3% plasticizer. Biological replicates n=10 for CsgA, n=15 for CL1 and n=20 for CL2. Data represented as mean±standard deviation. *p=0.01 (FIG. 3D), *p=0.0295. ****p<0.0001 (FIG. 3E). One-way ANOVA followed by Tukey's multiple comparisons test. FIG. 3F shows representative photographs showing tensile test of CL1 film with the lateral dimension of 0.5 cm by 4 cm. Left image: initial. Right image: before break. FIG. 3G shows plot of normalized Congo Red absorbance and the weights of wet cell pellets. For Congo Red absorbance, biological replicates n=3 for Sham, and n=6 for CsgA, CL1 and CL2. For weights of wet cell pellets, biological replicates n=3 for Sham, and n=10 for CsgA, CL1 and CL2. FIG. 3H shows plot of estimated wet weight of Curli nanofibers and the wet weight percentage of estimated Curli nanofibers to the cell pellet. Biological replicates n=6 for wet weight of Curli nanofibers and n=3 for the percentage weight. Data represented as mean±standard deviation. FIG. 3I shows Field Emission Scanning Electron Microscopy (FESEM) images of CsgA, CL1 and CL2. Top row: cell cultures. Scale bar 1 μm. Middle row: Top view of MECHS. Scale bar 10 μm. Bottom row: Side view of MECHS. Scale bar 10 μm.

FIGS. 4A-4J shows compostability, water-dispersibility and healability of MECHS. Representative photographs showing the biodegradation of MECHS and toilet paper in (FIG. 4A) a fresh fishnure and (FIG. 4B) a dry fishnure. The lateral dimensions of the MECHS film and the toilet paper were 5 cm by 5 cm. FIG. 4C plot shows the normalized biodegradation weight loss of MECHS (CsgA, CL1 and CL2), toilet paper, Kimwipe (KW), polyvinyl alcohol-McKesson (PVA-Mc), cellulose acetate (CA), poly-L-lactic acid (PLLA), polyethylene terephthalate (PET) and low density polyethylene (LDPE). Biological replicates n=3 for CsgA, CL1 and CL2. Technical replicates n=3 for paper, KW, PVA-Mc, CA, PLLA, PET and LDPE. Data represented as mean±standard deviation. Photographs show the dissolution of MECHS (FIG. 4D) toilet paper (FIG. 4E), PVA-Mc (FIG. 4F) and polyvinyl alcohol-Superpunch (PVA-Sp) (FIG. 4G). FIGS. 4D-4G shows lateral dimension of the films was 1 cm by 5 cm. FIG. 4H shows photograph of a black bean seedling grown in the soil mixed with fishnure (comprising biodegraded MECHS) in 9:1 ratio. FIG. 4I shows FESEM image of MECHS film healed by placing microliters of water at the site of abrasion (black arrows). Scale bar 200 μm. FIG. 4J photograph shows the MECHS films welded (black arrows) by using water. Scale bar 0.5 cm.

FIGS. 5A-5D show prototypes and mechanical landscape of MECHS. FIG. 5A photograph shows a refined prototype of MECHS film with a lateral dimension of 5 cm by 50 cm. FIG. 5B photograph shows the optical transparency of MECHS film with a lateral dimension of 10 cm by 15 cm. FIG. 5C shows photograph of a detergent pod (lateral dimension of 4 cm by 3 cm) wrapped with a MECHS film. FIG. 5D Ashby plot shows the Young's modulus and elongation at break for MECHS and various synthetic materials and biomaterials. Low density polyethylene (LDPE), Polytetrafluoroethylene (PTFE), Poly-L-lactic acid (PLLA), Polyethylene terephthalate (PET), Cellulose acetate (CA), Polypropylene (PP), Polyvinyl chloride (PVC), Polyvinyl alcohol-Superpunch (PVA-Sp), Polyvinyl alcohol-McKesson (PVA-Mc), Aluminum foil (Al Foil), Parafilm, Kimwipes (KW) and Toilet paper.

FIGS. 6A-6E show gelator treated Curli biomass. Photographs of Curli biomass obtained from 1% (FIG. 6A), 2% (FIG. 6B), 3% (FIG. 6C), 4% (FIG. 6D), and 5% gelator (FIG. 6E) (sodium dodecyl sulfate).

FIGS. 7A-7F show gelator treated MECHS. Photographs of MECHS obtained from 0% (FIG. 7A), 1% (FIG. 7B), 2% (FIG. 7C), 3% (FIG. 7D), 4% (FIG. 7E), and 5% gelator (FIG. 7F) (sodium dodecyl sulfate). Scale bar 1 cm.

FIGS. 8A-8E show plasticizer treated Curli biomass. Photographs of Curli biomass obtained from 1% (FIG. 8A), 2% (FIG. 8B), 3% (FIG. 8C), 4% (FIG. 8AD, and 5% plasticizer (FIG. 8E) (glycerol).

FIGS. 9A-9B show plasticizer treated MECHS. FIGS. 9A-9B show photographs of MECHS film obtained by treating with 3% plasticizer (glycerol) and casting on a silicone mold. Scale bar 1 cm.

FIGS. 10A-10F show tensile tests of MECHS. Stress strain curves of MECHS films obtained from 0% (FIG. 10A), 1% (FIG. 10B), 2% (FIG. 10C), 3% (FIG. 10D), 4% (FIG. 10E), and 5% plasticizer (FIG. 10F) (glycerol).

FIG. 11 shows tensile test of MECHS. Representative photographs show the tensile test of MECHS film with the lateral dimension of 1 cm by 4 cm obtained from 3% of gelator. Left image: initial. Right image: before break

FIGS. 12A-12B show tensile tests of MECHS. Stress strain curves of MECHS obtained from CL1 (FIG. 12A) and CL2 (FIG. 12B).

FIGS. 13A-13B show tensile tests of MECHS. FIG. 13A shows ultimate tensile strength of CsgA, CL1 and CL2 with 3% plasticizer. Data represented as mean±standard deviation. Biological replicates n=10 for CsgA, n=15 for CL1 and n=20 for CL2. **p=0.0042, ^{ns}p=0.0637. One-way ANOVA followed by Tukey's multiple comparisons test. FIG. 13B shows representative photographs show the tensile test of CL2 film with the lateral dimension of 0.5 cm by 4 cm. Left image: initial. Right image: before break.

FIGS. 14A-14B show Congo Red assay. FIG. 14A shows Congo Red absorbance at 480 nm for various samples. FIG. 14B shows Congo Red standard curve for purified CsgA (wet weight, CsgA-His). Biological replicates n=3 for Sham and n=6 for CsgA, CL1 and CL2. The net Congo Red absorbance of Curli in CsgA, CL1 and CL2 were determined by subtracting the absorbance values of cell pellet having a sham plasmid (without Curli operon), to account for the non-specific binding to other biomolecules. Data represented as mean±standard deviation.

FIGS. 15A-15B show weight analysis. FIG. 15A shows weight of wet and dry pellet of Curli biofilm obtained from 500 mL cultures. FIG. 15B shows percentage of dry to wet pellet weight of Curli biofilm. n=5. Data represented as mean±standard deviation.

FIGS. 16A-16D show weight analysis of gelator treated MECHS. Weight of MECHS films (FIG. 16A) and the dried supernatant (FIG. 16B) obtained from 1 to 5% of gelator. Percentage of MECHS film weight to dry Curli biofilm pellet (FIG. 16C) weight (20.3% of wet pellet) and dry weight of all precursors (FIG. 16D) for 1 to 5% of gelator. All precursors correspond to weights of cell pellet and that of 1-5% gelator. n=4. Data represented as mean±standard deviation.

FIGS. 17A-17E show weight analysis of gelator treated MECHS. Weights of wet pellet, MECHS film, dried supernatant, experimental net (MECHS and dried Supernatant) and calculated theoretical net (20.3% of wet pellet) for 1% (FIG. 17A), 2% (FIG. 17B), 3% (FIG. 17C), 4% (FIG. 17D), and 5% (FIG. 17E) of gelator. n=4. Data represented as mean±standard deviation.

FIG. 18 shows compositional analysis of MECHS. Energy Dispersive X-ray Analysis (EDAX) of E. coli Curli biofilm cell pellets pretreated with or without 3% gelator (SDS) shows the weight percentage of various elements. n=3. Data represented as mean±standard deviation.

FIGS. 19A-19D show weight analysis of plasticizer treated MECHS. Weight of MECHS films (FIG. 19A) and the dried supernatant (FIG. 19B) obtained from 1 to 5% of plasticizer. Percentage of MECHS film weight to dry Curli biofilm pellet (FIG. 19C) weight (20.3% of wet pellet) and dry weight of all precursors (FIG. 19D) for 1 to 5% of plasticizer. All precursors correspond to weights of cell pellet and that of 3% gelator and 1-5% plasticizer. n=4. Data represented as mean±standard deviation.

FIGS. 20A-20E show weight analysis of plasticizer treated MECHS. Weights of wet pellet, MECHS film, dried supernatant, experimental net (MECHS and dried Supernatant) and calculated theoretical net (20.3% of wet pellet) for 1% (FIG. 20A), 2% (FIG. 20B), 3% (FIG. 20C), 4% (FIG. 20D), and 5% (FIG. 20E) of plasticizer. n=4. Data represented as mean±standard deviation.

FIGS. 21A-21D show weight analysis of covalently crosslinked MECHS. Weight of MECHS films (FIG. 20A) and the dried supernatant (FIG. 20B) of CsgA, CL1 and CL2. Percentage of MECHS weight to dry Curli biofilm pellet (FIG. 20C) weight (20.3% of wet pellet) and dry weight of all precursors (FIG. 20D) for CsgA, CL1 and CL2. All precursors correspond to weights of cell pellet and that of 3% gelator and 3% plasticizer. n=4. Data represented as mean±standard deviation.

FIGS. 22A-22C show weight analysis of covalently crosslinked MECHS. Weights of wet pellet, MECHS, dried supernatant, experimental net (MECHS and dried Supernatant) and calculated theoretical net (20.3% of wet pellet) for CL1 (FIG. 21A) and CL2 (FIG. 21B). n=4. Data represented as mean±standard deviation.

FIGS. 23A-23B show Morphological analysis of MECHS. FESEM image of top view (FIG. 23A) and side view (FIG. 23B) of MECHS film obtained from 3% gelator. Scale bar 20 μm and 10 μm, respectively.

FIGS. 24A-24H show morphological analysis of MECHS. FESEM image of top view (FIGS. 24A, 24C, 24E, and 24G) and side view (FIGS. 24B, 24D, 24F, and 24H) of MECHS film obtained from 1% (FIGS. 24A, 24B), 2% (FIGS. 24C, 24D), 4% (FIGS. 24E, 24F), and 5% (FIGS. 24G, 24H) plasticizer. Scale bar Top Row: 20 μm and Bottom Row: 10 μm.

FIG. 25 shows greenhouse for biodegradation experiment. Photograph of a mini greenhouse setup utilized for testing the compostability of MECHS.

FIG. 26 shows compostability of MECHS and Kimwipe in a fresh fishnure. Photographs show the biodegradation of CL1, CL2 and Kimwipe in a fresh fishnure. The lateral dimensions of the samples were 5 cm by 5 cm.

FIG. 27 shows compostability of Bioplastics and Plastics in a fresh fishnure. Photographs show the biodegradation of cellulose acetate (CA), poly-L-lactic acid (PLLA), low density polyethylene (LDPE) and polyethylene terephthalate (PET) in a fresh fishnure. The lateral dimensions of the samples were 5 cm by 5 cm.

FIG. 28 shows compostability of Polyvinyl alcohol in a fresh fishnure. Photographs show the films of Polyvinyl alcohol-McKesson (PVA-Mc) and Polyvinyl alcohol-Superpunch (PVA-Sp) in a fresh fishnure. The lateral dimensions of the samples were 5 cm by 5 cm.

FIG. 29 shows compostability of MECHS and Kimwipe in a dry fishnure. Photographs show the biodegradation of CL1 and Kimwipe in a dry fishnure. The lateral dimensions of the samples were 5 cm by 5 cm.

FIG. 30 show compostability of Bioplastics and Plastics in a dry fishnure. Photographs show the biodegradation of Polyvinyl alcohol-McKesson (PVA-Mc) and Polyvinyl alcohol-Superpunch (PVA-Sp), cellulose acetate (CA), poly-L-lactic acid (PLLA), low density polyethylene (LDPE) and polyethylene terephthalate (PET) in a dry fishnure. The lateral dimensions of the samples were 5 cm by 5 cm.

FIGS. 31A-31D show biofabrication of large MECHS prototype. Photographs show casting of Curli biomass on to a silicone mold (FIG. 31A), ambient dried MECHS (FIG. 31B), peeling of MECHS film from the silicone mold (FIG. 31C), and free-standing flexible MECHS film (FIG. 31D) of lateral dimension 10 cm by 25 cm.

FIGS. 32A-32C show tensile tests. Stress strain curves of Low-density polyethylene (LDPE) (FIG. 32A), Polyvinyl chloride (PVC) (FIG. 32B), and Polytetrafluoroethylene (PTFE) (FIG. 32C).

FIGS. 33A-33B show tensile tests. Stress strain curves of Polyethylene terephthalate (PET) (FIG. 33A) and Polypropylene (PP) (FIG. 33B).

FIGS. 34A-34B show tensile tests. Stress strain curves of PVA-McKesson (FIG. 34A) and PVA-Superpunch (FIG. 34B). PVA: Polyvinyl alcohol.

FIGS. 35A-35B show tensile tests. Stress strain curves of Poly-L-lactic acid (PLLA) (FIG. 35A) and Cellulose Acetate (FIG. 35B).

FIGS. 36A-36B show tensile tests. Stress strain curves of Aluminum foil (FIG. 36A) and parafilm (FIG. 36B).

FIGS. 37A-37B show tensile tests. Stress strain curves of Toilet paper (FIG. 37A) and Kimwipe (FIG. 37B).

FIG. 38 shows tensile tests. Plot shows the elongation at break for MECHS, various synthetic materials and biomaterials. Biological replicates n=10 for 0-5% plasticizer, n=15 for CL1, n=20 for CL2 and n=5 for all other samples. Low density polyethylene (LDPE), Polytetrafluoroethylene (PTFE), Poly-L-lactic acid (PLLA), Polyethylene terephthalate (PET), Cellulose acetate (CA), Polypropylene (PP), Polyvinyl chloride (PVC), Polyvinyl alcohol-Superpunch (PVA-Sp), Polyvinyl alcohol-McKesson (PVA-Mc), Aluminum foil (Al Foil), Parafilm, Kimwipes (KW) and Toilet paper.

FIG. 39 shows tensile tests. Plot shows the Young's modulus for MECHS, various synthetic materials and biomaterials. Biological replicates n=10 for 0-5% plasticizer, n=15 for CL1, n=20 for CL2 and n=5 for all other samples. Low density polyethylene (LDPE), Polytetrafluoroethylene (PTFE), Poly-L-lactic acid (PLLA), Polyethylene terephthalate (PET), Cellulose acetate (CA), Polypropylene (PP), Polyvinyl chloride (PVC), Polyvinyl alcohol-Superpunch (PVA-Sp), Polyvinyl alcohol-McKesson (PVA-Mc), Aluminum foil (Al Foil), Parafilm, Kimwipes (KW) and Toilet paper.

FIG. 40 shows tensile tests. Plot shows the ultimate tensile strength for MECHS, various synthetic materials and biomaterials. Biological replicates n=10 for 0-5% plasticizer, n=15 for CL1, n=20 for CL2 and n=5 for all other samples. Low density polyethylene (LDPE), Polytetrafluoroethylene (PTFE), Poly-L-lactic acid (PLLA), Polyethylene terephthalate (PET), Cellulose acetate (CA), Polypropylene (PP), Polyvinyl chloride (PVC), Polyvinyl alcohol-Superpunch (PVA-Sp), Polyvinyl alcohol-McKesson (PVA-Mc), Aluminum foil (Al Foil), Parafilm, Kimwipes (KW) and Toilet paper.

### DETAILED DESCRIPTION

Advanced design strategies are essential to realize the full potential of engineered living materials, including their biodegradability, manufacturability, sustainability, and ability to tailor functional properties. Toward these goals, we present mechanically engineered living material with compostability, healability, and scalability—a material that integrates these features in the form of a stretchable plastic that is simultaneously flushable, compostable, and exhibits the characteristics of paper. This plastic/paper-like material is produced in scalable quantities (0.5-1 g L⁻¹), directly from cultured bacterial biomass (40%) containing engineered Curli protein nanofibers. The elongation at break (1-160%) and Young's modulus (6-450 MPa) is tuned to more than two orders of magnitude. By genetically encoded covalent crosslinking of Curli nanofibers, we increase the Young's modulus by two times. The designed engineered living materials biodegrade completely in 15-75 days, while its mechanical properties are comparable to petrochemical plastics and thus may find use as compostable materials for primary packaging.

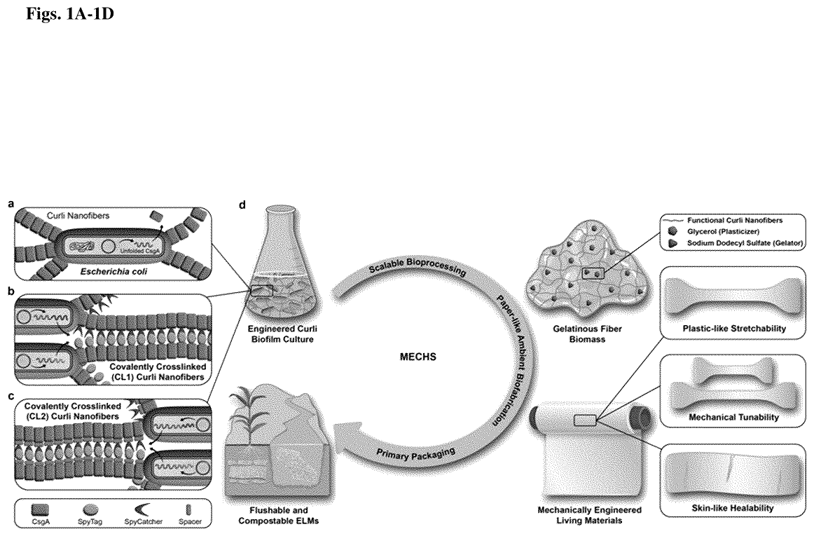

The emerging field of Engineered Living Materials (ELMs) employs synthetic biology design principles to harness the programmability and the manufacturing capabilities of living cells to produce functional materials. ELMs research not only provides avenues to integrate life-like properties into materials but also aims to realize de novo functionalities that are not found in natural or synthetic materials. In recent years, several ELMs have been developed to demonstrate various functionalities such as adhesion, catalysis, mineralization, remediation, wound healing, and therapeutics. ELMs that are mechanically stiff or soft have also been reported but the rational modulation of mechanical properties to a wide range through genetic programming remains elusive. In this regard, we present an ELM called MECHS, which stands for Mechanically Engineered Living Material with Compostability, Healability, and Scalability (FIG. 1).

Advances in biomanufacturing are important at a time when human-made materials have been estimated to outweigh all the living biomass of planet Earth. The existing paradigm of a linear materials economy (make-use-dispose) for synthetic materials is causing potentially irreversible damage to our ecosystem in the form of pollution and global warming. While many strategies will need to be employed to address these challenges, bio-based manufacturing will need to be part of the solution. Inspired by natural systems that utilize sustainable feedstocks and energy-efficient processes, coupled with their biodegradation to initiate a new cycle, biomanufacturing should strive to create materials that have similar recyclability or potential for conversion to benign components to create a circular material economy. Such sustainable solutions enabled by biomanufacturing will also make inroads toward practical implementation through a combination of appropriate government policies, public interest, and investment.

We produced a bioplastic known as AquaPlastic composed of recombinant protein nanofibers produced by E. coli. It exhibited a Young's modulus of ˜1 GPa and ultimate tensile strength of ˜25 MPa, comparable to petrochemical plastics and other bioplastics. AquaPlastic was also resistant to various chemicals (e.g., acid, base, and organic solvents), and could adhere to and coat a wide range of surfaces, protecting them from wear and environmental conditions. However, the broad utility of AquaPlastic was limited due to its brittleness and lack of scalability. Additionally, we showed that whole microbial biomass could be dried to form cohesive and glassy stiff materials with a streamlined fabrication and higher yields compared to AquaPlastic, at the expense of tunability.

We report, herein, a fabrication strategy to combine whole cellular biomass and engineered extracellular matrix protein nanofibers that facilitates tuning of their mechanical properties. Our material, MECHS, exhibits properties similar to both plastic and paper, showcasing: 1) a fabrication strategy that enables larger scale production of flexible films at ambient conditions, analogous to paper manufacturing; 2) genetic engineering to tailor their tensile stiffness and strength; 3) compositional and morphological analysis; 4) compostability, 5) a landscape of achievable mechanical properties comparable to conventional petrochemical plastics, bioplastics and other relevant bio- and synthetic materials; and, 6) prototypes for disposable packaging applications, contributing to the creation of a sustainable circular material economy.

In one aspect the present disclosure provides an engineered fiber, comprising a first Curli protein fused to a first binding peptide and a second Curli protein fused to a second binding peptide. The first binding peptide may be covalently bound to the second binding peptide, crosslinking the first Curli protein with the second Curli protein.

### Definitions

For convenience, certain terms employed in the specification, examples, and appended claims are collected here.

As used herein, the singular forms “a,” “an” and “the” are intended to include the plural forms as well, unless the context clearly indicates otherwise.

The term “about” means within an acceptable error range for the particular value as determined by one of ordinary skill in the art, which will depend in part on how the values measured or determined, i.e., the limitations of the measurement system. Where the terms “about” or “approximately” are used in the context of compositions containing amounts of ingredients or conditions such as temperature, these values include the stated value with a variation of 0-10% around the value (X±10%).

The terms “including,” “includes,” “having,” “has,” “with,” or variants thereof are inclusive in a manner similar to the term “comprising.” The term “consisting” and the grammatical variations of consist encompass embodiments with only the listed elements and excluding any other elements. The phrases “consisting essentially of” or “consists essentially of” encompass embodiments containing the specified materials or steps and those including materials and steps that do not materially affect the basic and novel characteristic(s) of the embodiments.

Ranges are stated in shorthand to avoid having to set out at length and describe each and every value within the range. Therefore, when ranges are stated for a value, any appropriate value within the range can be selected, and these values include the upper value and the lower value of the range. For example, a range of two to thirty represents the terminal values of two and thirty, as well as the intermediate values between two to thirty, and all intermediate ranges encompassed within two to thirty, such as two to five, two to eight, two to ten, etc.

The terms “microbial cell,” “microorganism,” and “microbe” are used interchangeably and should be interpreted to encompass microscopic organism, particularly those commonly studied by microbiologists. Such organisms may include, but are not limited to, bacteria, fungi, and other single-celled organisms including the non-limiting examples of archaea, protozoa, fungi, algae, green algae, rotifers, planarians, and parasitic pathogens. Preferably, the microbes may be bacteria.

As used herein, the term “engineered microbial cell,” “engineered microorganism,” and “engineered microbe” refer to a microbial cell that has been genetically modified from its native state. For instance, an engineered microbial cell may have nucleotide insertions, nucleotide deletions, nucleotide rearrangements, and nucleotide modifications introduced into their DNA. These genetic modifications may be stably incorporated into the genome of the microbe (e.g., present in the chromosome of a bacteria or bacterial cell), or on an exogenous nucleic acid, such as a plasmid in a bacteria or bacterial cell. Accordingly, engineered microbial cells of the disclosure may comprise exogenous nucleotide sequences on plasmids. Alternatively, recombinant microbial cells may comprise exogenous nucleotide sequences stably incorporated into their genome. In some embodiments, the engineered microbe is non-pathogenic. In some embodiments, the engineered microbe is pathogenic. As used herein, the term “expression” refers to the transcription and stable accumulation of sense (mRNA) or anti-sense RNA derived from a nucleic acid, to translation of an mRNA into a polypeptide, and/or the final product encoded by a gene or fragment thereof.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising at least one amino acid catabolism enzyme operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

“Exogenous nucleic acid” refers to a nucleic acid, DNA, or RNA, which has been artificially introduced into a cell. Such exogenous nucleic acid may or may not be a copy of a sequence or fragments thereof which is naturally found in the cell into which it was introduced.

“Endogenous nucleic acid” refers to a nucleic acid, gene, polynucleotide, DNA, RNA, mRNA, or cDNA molecule that is naturally present in a microorganism. An endogenous sequence is “native” to, i.e., indigenous to, the microorganism.

A “chimeric nucleic acid” comprises a first nucleotide sequence linked to a second nucleotide sequence, wherein the second nucleotide sequence is different from the sequence which is associated with the first nucleotide sequence in cells in which the first nucleotide sequence occurs naturally.

A constitutive promoter expresses an operably linked gene when RNA polymerase holoenzyme is available. Expression of a gene under the control of a constitutive promoter does not depend on the presence of an inducer.

An inducible promoter expresses an operably linked gene only in the presence of an inducer. An inducer activates the transcription machinery that induces the expression of a gene operably linked to an inducible promoter.

### Engineered Fiber

The Curli protein is a type of amyloid fiber produced by certain strains of enterobacteria. They are extracellular fibers located on bacteria such as E. coli and Salmonella spp. These fibers serve to promote cell community behavior through biofilm formation in the extracellular matrix. The Curli pili are generally assembled through the extracellular nucleation precipitation pathway.

A convenient way to identify genes important for Curli production is by growing curliated bacteria on plates supplemented with Congo red diazo dye, which causes a red shift. Curli fibers, and thus the Curli protein, are coded by two divergently-transcribed operons in E. coli: the csgBAC and csgDEFG operons. In total, these operons encode at least seven proteins. The agfBA and agfDEFG operons, which have been identified in Salmonella spp., are homologous to the csgBAC and csgDEFG operons. The csgBAC operon is responsible for the coding the three proteins CsgB, CsgA, and CsgC—all responsible for either the major subunit formation within the Curli fiber or the inhibition of it. The csgDEFG operon codes for proteins CsgD, CsgE, CsgF, and CsgG, and is responsible for the assembly, translocation, and regulation of the Curli protein. CsgD is a positive transcriptional regulator of the csgBAC operon. The csgBA operon encodes the major structural subunit of Curli, CsgA, as well as the nucleator protein, CsgB.

Curli is a major component in the biofilm generated by gram-negative bacteria such as E. coli and Salmonella spp. These biofilms allow gram-negative bacteria to better colonize in a given environment, protecting them from oxidative stress and dehydration. Curli proteins and biofilms alike are very resistant to chemical stressors to a point where stronger pretreatment is required in order for Curli to degrade or dissolve in sodium dodecyl sulphate (SDS).

### CsgA

CsgA is the major subunit of the Curli protein and weighs approximately 13.1 kilodalton. This protein consists of three domains which have a tendency to aggregate and form amyloid fibrils: a single peptide, a 22-amino acid N-terminal sequence (used for secretion), and an amyloid core domain at the C-terminal sequence. The amyloid core domain is composed of 5 repeating (yet not exact) sequences revolving around the sequence: Ser-X5-Gln-X-Gly-X-Gly-Asn-X-Ala-X3-Gln (SEQ ID NO: 19). This repeating sequence is the characteristic subunit that forms its aggregatable β-sheet.

An example of CsgA is provided by the sequence of SEQ ID NO: 1. Homologues of CsgA from microorganisms can be used in the microorganisms and methods described herein. Non-limiting examples of the homologs of CsgA in the instant invention are represented by UniProt entries: P28307, P21158, POA1E7, POA1E5, POA1E6, Q93U24, P54379, A5HDN1, Q89JI3, and Q53656.

A binding peptide as disclosed herein refers to a peptide capable of binding to another binding peptide via a covalent link. The first and second binding peptide may be any one of the following pairs: SpyCatcher-Spytag, SnoopCatcher-Snooptag, and streptavidin-biotin.

The SpyTag/SpyCatcher system is a technology for irreversible conjugation of recombinant proteins. The peptide SpyTag (13 amino acids) spontaneously reacts with the protein SpyCatcher (12.3 kDa) to form an intermolecular isopeptide bond between the pair. DNA sequence encoding either SpyTag or SpyCatcher can be recombinantly introduced into the DNA sequence encoding a protein of interest, forming a fusion protein. These fusion proteins can be covalently linked when mixed in a reaction through the SpyTag/SpyCatcher system. The Spytag may comprise the amino acid sequence set forth in SEQ ID NO: 3. The SpyCatcher may comprise the amino acid sequence set forth in SEQ ID NO: 4.

Similar to SpyTag/SpyCatcher, SnoopTag and SnoopCatcher form an isopeptide bond when mixed together. SnoopTag/SnoopCatcher react with each other. Streptavidin homo-tetramers have an extraordinarily high affinity for biotin.

The first Curli protein may be fused to the first binding peptide via a spacer and/or linker. Similarly, the second Curli protein may be fused to the second binding peptide via a spacer and/or linker. In some embodiments, the linker is glycine serine linker. In some embodiments, the glycine serine linker comprises the amino acid sequence set forth in any one of SEQ ID NOs: 2, 11, and 13-18 and the sequence GSG.

Exemplary glycine serine linkers includes, but not limited to, the ones represented by the following amino acid sequences or comparable equivalents, or their combinations, including their codon optimized nucleic acid sequences, fragments, mutants, variants, or functional analogs thereof:

(SEQ ID NO: 11)
GGGGSGGGGSGGGGS;
GSG
(SEQ ID NO: 13)
GSGG;
(SEQ ID NO: 14)
GGSGG;
(SEQ ID NO: 15)
GGSGGGGSGG;
(SEQ ID NO: 16)
GGSGGGGSGGGGSGG;
(SEQ ID NO: 17)
SGGSGG;
(SEQ ID NO: 18)
GGGGSGGGGS.

In some embodiments, the glycine serine linkers share at least 50% identity with the sequences described herein above or comparable equivalents, or a combination thereof, including their codon optimized nucleic acid sequences, fragments, mutants, variants, or functional analogs thereof.

Given the above amino acid sequences of the glycine serine linkers, a person skilled in the art would be able to deduce all possible DNA or RNA sequences that encodes the above glycine serine linkers. Such DNA or RNA sequences are deemed to be incorporated in this disclosure. In some embodiments, the glycine serine linker is encoded by a glycine serine linker sequence which may be either a DNA, an RNA, or an mRNA.

In some embodiments, the spacer comprises an intrinsically disordered protein. An intrinsically disordered protein (IDP) is a protein that lacks a fixed or ordered three-dimensional structure, typically in the absence of its macromolecular interaction partners, such as other proteins or RNA. IDPs range from fully unstructured to partially structured and include random coil, molten globule-like aggregates, or flexible linkers in large multi-domain proteins.

Examples of IDPs: TDP-43, FUS, SMN, ataxin-2, optineurin, angiogenin, tau, α-synuclein, huntingtin, DRPLA protein, elastin, and casein.

In some embodiments, the casein comprises the amino acid sequence set forth in SEQ ID NO: 5.

In some embodiments, the fiber is a nonfiber. Nanofibers are fibers with diameters in the nanometer range (typically, between 1 nm and 1 m).

### Engineered Living Material (ELM)

The Curli nanofiber-based bioplastic fabrication protocol (i.e., AquaPlastic), which involved the filtration of bacterial culture to concentrate Curli nanofibers and form gels was reported (Duraj-Thatte, A. M. et al. Nat. Chem. Biol. 17, 732-738 (2021)). Using that protocol, concerns about clogging necessitated the use of filters with 10 μm pores, leading to the loss of significant amounts of Curli nanofibers. The MECHS fabrication protocol disclosed herein increased the yield of bioplastic by a factor of ten by utilizing not only all the Curli nanofibers in the pelletized biomass, but also the other water insoluble cellular biomass. We also found that the SDS gelator could be supplemented with a plasticizer like glycerol to obtain flexible films of MECHS, as compared to the significantly more brittle AquaPlastic. Glycerol being a byproduct of the biodiesel industry offers several advantages viz., nontoxic, low-cost, and renewable. Unlike the conventional petrochemical plastics and other bioplastics that are processed by thermal molding, MECHS was molded into films by ambient drying of gelatinous biomass, which we have termed it as aquamolding. The healing and welding of MECHS films by using tiny droplets of water are termed as aquahealing and aquawelding, respectively.

The tunability of MECHS, with its range of mechanical properties (e.g., elongation at break 1-160%; Young's modulus 6-450 MPa) and transparency, provides a promising platform to access biodegradable alternatives to synthetic materials like petrochemical plastics. We were also able to use our streamlined protocol to achieve high yields of 0.5-1 g L⁻¹ and generate large, refined prototypes. Further, we could obtain ˜40 cm² of MECHS film per liter of culture. Therefore, to biofabricate a roll of MECHS film with the lateral dimensions 2 m×5 cm, we would require ˜25 L of culture. Another notable feature of this work is that 40% of the total cellular biomass gets incorporated into the plastic/paper-like MECHS, which could utilize cellular biomass for the development of various sustainable functional materials.

During the MECHS biofabrication, most of the SDS ends up in the supernatant, which when dried resulted in a brown-yellowish color pellet. Thus, we believe that SDS being a surfactant removes the brown-yellowish color of the cell pellet, which makes MECHS film transparent. Curli nanofibers are assembled from CsgA protein building blocks that comprises of a rigid beta-helical structure, which in simple terms can be regarded as a quasi-crystalline ordering. So, when these Curli nanofibers (without plasticizer) based rigid materials are subjected to tensile stress beyond its yield point, the strain induced crack, propagates and it quickly breaks the material. But by incorporating a plasticizer like glycerol, the amorphous nature of plasticizer that surrounds the rigid Curli nanofibers inhibit the crack propagation by subjecting the material to undergo plastic deformation. Thus, with increasing plasticizer amounts from 1-5%, the elongation at break was found to increase from 1 to 160%.

Plastics are one of the most abundant human-made materials, with over 8.3 billion tons produced cumulatively, 79% of which are estimated to have accumulated in landfills and oceans. In addition, the contamination of microplastics in almost all parts of the globe further enhances their threat to our health and the environment. Biodegradable bioplastics account for less than 1% of the global plastic market and their limited properties warrants the development of alternatives. Given that the typical lifetime of a packaging material is 1-2 years, and the packaging industry accounts for nearly one third of the plastic market, there exists a tremendous scope and opportunity for biodegradable packaging, though success will likely need to be achieved through the commercialization of drop-in replacements for existing materials. Notably, water soluble polymers like PVA (commonly found in detergent pods) have limited biodegradation under diverse settings of land and water. In many cases, dissolvable polymers like PVA are blended with petrochemical plastics to enhance certain material properties but this limits their water dispersibility and biodegradability (as observed in our biodegradation tests with the commercially available PVA-Mc).

We were able to develop refined prototypes of MECHS thin films. This can improve the mechanical properties (e.g., ultimate tensile strength, tear strength) and resistance to water. Furthermore, the circular materials economy loop will have to be closed by employing a feedstock for bacterial culture derived closely from CO₂ fixation, such as cellulose hydrolysate obtained from agricultural waste. There are also several opportunities to utilize the synthetic biology tools to tailor the material properties of Curli nanofibers. The biodegraded MECHS may be used as a biofertilizer for plant growth. We have demonstrated that the manufacturing capabilities of living cells can be employed to produce the mechanically tunable, scalable, and compostable ELMs as a potential alternative to synthetic materials like plastics. Finally, we believe that innovative approaches involving synthetic biology and materials engineering could lead to greater advancements in creating energy efficient and sustainable solutions to a greener ecosystem.

The plastic-like material named MECHS can be produced to about 0.5 to 1 g per liter directly from the Curli biofilm culture, with nearly 10 times higher yield than AquaPlastic. We show that the elongation at break can be tailored to more than two orders of magnitude. By genetically encoding the covalent crosslinking of Curli nanofibers, we increase the Young's modulus and ultimate tensile strength by two times. MECHS biodegrades completely in 2 weeks in fresh organic humus compost, whereas in dry settings, it takes 10 weeks, which is comparable to that of paper. The mechanical properties of MECHS are comparable to various synthetic materials like petrochemical plastics and thus may find use as compostable primary packages.

### Engineered Microbial Cells

Disclosed herein are engineered microbial cells comprising an exogenous nucleic acid encoding the engineered fiber described herein. The engineered fiber may comprise a first Curli protein fused to a first binding peptide and a second Curli protein fused to a second binding peptide, wherein the first binding peptide is covalently bound to the second binding peptide, crosslinking the first Curli protein with the second Curli protein. In some embodiments, the engineered microbial cell is a bacterial or a fungal cell. In some embodiments, the engineered microbial cell is Escherichia coli, Lactobacillus rhamnosus, Salmonella spp, or Saccharomyces cerevisiae. In some embodiments, the engineered microbial cell is Escherichia coli strain PQN4.

In some embodiments, the engineered microbial cell is bacterium such as Turicibacter sanguinis, Escherichia coli, or Bacteroides thetaiotaomicron.

In some embodiments, the engineered microorganism is Abiotrophia defectiva, Abiotrophia para_adiacens, Abiotrophia sp. oral clone P4PA_155 P1, Acetanaerobacterium elongatum, Acetivibrio cellulolyticus, Acetivibrio ethanolgignens, Acetobacter aceti, Acetobacter fabarum, Acetobacter lovaniensis, Acetobacter malorum, Acetobacter orientalis, Acetobacter pasteurianus, Acetobacter pomorum, Acetobacter syzygii, Acetobacter tropicalis, Acetobacteraceae bacterium AT_5844, Acholeplasma laidlawii, Achromobacter denitrificans, Achromobacter piechaudii, Achromobacter xylosoxidans, Acidaminococcus fermentans, Acidaminococcus intestini, Acidaminococcus sp. D21, Acidilobus saccharovorans, Acidithiobacillus ferrivorans, Acidovorax sp. 98_63833, Acinetobacter baumannii, Acinetobacter calcoaceticus, Acinetobacter genomosp. C1, Acinetobacter haemolyticus, Acinetobacter johnsonii, Acinetobacter junii, Acinetobacter lwoffii, Acinetobacter parvus, Acinetobacter radioresistens, Acinetobacter schindleri, Acinetobacter sp. 56A1, Acinetobacter sp. CIP 101934, Acinetobacter sp. CIP 102143, Acinetobacter sp. CIP 53.82, Acinetobacter sp. M16_22, Acinetobacter sp. RUH2624, Acinetobacter sp. SH024, Actinobacillus actinomycetemcomitans, Actinobacillus minor, Actinobacillus pleuropneumoniae, Actinobacillus succinogenes, Actinobacillus ureae, Actinobaculum massiliae, Actinobaculum schaalii, Actinobaculum sp. BM #101342, Actinobaculum sp. P2P_19 P1, Actinomyces cardiffensis, Actinomyces europaeus, Actinomyces funkei, Actinomyces genomosp. C1, Actinomyces genomosp. C2, Actinomyces genomosp. P1 oral clone MB6_C03, Actinomyces georgiae, Actinomyces israelii, Actinomyces massiliensis, Actinomyces meyeri, Actinomyces naeslundii, Actinomyces nasicola, Actinomyces neuii, Actinomyces odontolyticus, Actinomyces oricola, Actinomyces orihominis, Actinomyces oris, Actinomyces sp. 7400942, Actinomyces sp. c109, Actinomyces sp. CCUG 37290, Actinomyces sp. ChDC B197, Actinomyces sp. GEJ15, Actinomyces sp. HKU31, Actinomyces sp. ICM34, Actinomyces sp. ICM41, Actinomyces sp. ICM47, Actinomyces sp. ICM54, Actinomyces sp. M2231_94_1, Actinomyces sp. oral clone GU009, Actinomyces sp. oral clone GU067, Actinomyces sp. oral clone 10076, Actinomyces sp. oral clone 10077, Actinomyces sp. oral clone IP073, Actinomyces sp. oral clone IP081, Actinomyces sp. oral clone JA063, Actinomyces sp. oral taxon 170, Actinomyces sp. oral taxon 171, Actinomyces sp. oral taxon 178, Actinomyces sp. oral taxon 180, Actinomyces sp. oral taxon 848, Actinomyces sp. oral taxon C55, Actinomyces sp. TeJ5, Actinomyces urogenitalis, Actinomyces viscosus, Adlercreutzia equolifaciens, Aerococcus sanguinicola, Aerococcus urinae, Aerococcus urinaeequi, Aerococcus viridans, Aeromicrobium marinum, Aeromicrobium sp. JC14, Aeromonas allosaccharophila, Aeromonas enteropelogenes, Aeromonas hydrophila, Aeromonas jandaei, Aeromonas salmonicida, Aeromonas trota, Aeromonas veronii, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, Afipia genomosp. 4, Aggregatibacter actinomycetemcomitans, Aggregatibacter aphrophilus, Aggregatibacter segnis, Agrobacterium radiobacter, Agrobacterium tumefaciens, Agrococcus jenensis, Akkermansia muciniphila, Alcaligenes faecalis, Alcaligenes sp. CO14, Alcaligenes sp. S3, Alicyclobacillus acidocaldarius, Alicyclobacillus acidoterrestris, Alicyclobacillus contaminans, Alicyclobacillus cycloheptanicus, Alicyclobacillus herbarius, Alicyclobacillus pomorum, Alicyclobacillus sp. CCUG 53762, Alistipes finegoldii, Alistipes indistinctus, Alistipes onderdonkii, Alistipes putredinis, Alistipes shahii, Alistipes sp. HGB5, Alistipes sp. JC50, Alistipes sp. RMA 9912, Alkaliphilus metalliredigenes, Alkaliphilus oremlandii, Alloscardovia omnicolens, Alloscardovia sp. OB7196, Anaerobaculum hydrogeniformans, Anaerobiospirillum succiniciproducens, Anaerobiospirillum thomasii, Anaerococcus hydrogenalis, Anaerococcus lactolyticus, Anaerococcus octavius, Anaerococcus prevotii, Anaerococcus sp. 8404299, Anaerococcus sp. 8405254, Anaerococcus sp. 9401487, Anaerococcus sp. 9403502, Anaerococcus sp. gpac104, Anaerococcus sp. gpac126, Anaerococcus sp. gpac155, Anaerococcus sp. gpac199, Anaerococcus sp. gpac215, Anaerococcus tetradius, Anaerococcus vaginalis, Anaerofustis stercorihominis, Anaeroglobus geminatus, Anaerosporobacter mobilis, Anaerostipes caccae, Anaerostipes sp. 3_2_56FAA, Anaerotruncus colihominis, Anaplasma marginale, Anaplasma phagocytophilum, Aneurinibacillus aneurinilyticus, Aneurinibacillus danicus, Aneurinibacillus migulanus, Aneurinibacillus terranovensis, Aneurinibacillus thermoaerophilus, Anoxybacillus contaminans, Anoxybacillus flavithermus, Arcanobacterium haemolyticum, Arcanobacterium pyogenes, Arcobacter butzleri, Arcobacter cryaerophilus, Arthrobacter agilis, Arthrobacter arilaitensis, Arthrobacter bergerei, Arthrobacter globiformis, Arthrobacter nicotianae, Atopobium minutum, Atopobium parvulum, Atopobium rimae, Atopobium sp. BS2, Atopobium sp. F0209, Atopobium sp. ICM42b10, Atopobium sp. ICM57, Atopobium vaginae, Aurantimonas coralicida, Aureimonas altamirensis, Auritibacter ignavus, Averyella dalhousiensis, Bacillus aeolius, Bacillus aerophilus, Bacillus aestuarii, Bacillus alcalophilus, Bacillus amyloliquefaciens, Bacillus anthracis, Bacillus atrophaeus, Bacillus badius, Bacillus cereus, Bacillus circulans, Bacillus clausii, Bacillus coagulans, Bacillus firmus, Bacillus flexus, Bacillus fordii, Bacillus gelatini, Bacillus halmapalus, Bacillus halodurans, Bacillus herbersteinensis, Bacillus horti, Bacillus idriensis, Bacillus lentus, Bacillus licheniformis, Bacillus megaterium, Bacillus nealsonii, Bacillus niabensis, Bacillus niacini, Bacillus pocheonensis, Bacillus pumilus, Bacillus safensis, Bacillus simplex, Bacillus sonorensis, Bacillus sp. 10403023 MM10403188, Bacillus sp. 2_A_57_CT2, Bacillus sp. 2008724126, Bacillus sp. 2008724139, Bacillus sp. 7_16AIA, Bacillus sp. 9_3AIA, Bacillus sp. AP8, Bacillus sp. B27(2008), Bacillus sp. BT1B_CT2, Bacillus sp. GB1.1, Bacillus sp. GB9, Bacillus sp. HU19.1, Bacillus sp. HU29, Bacillus sp. HU33.1, Bacillus sp. JC6, Bacillus sp. oral taxon F26, Bacillus sp. oral taxon F28, Bacillus sp. oral taxon F79, Bacillus sp. SRC_DSF1, Bacillus sp. SRC_DSF10, Bacillus sp. SRC_DSF2, Bacillus sp. SRC_DSF6, Bacillus sp. tc09, Bacillus sp. zh168, Bacillus sphaericus, Bacillus sporothermodurans, Bacillus subtilis, Bacillus thermoamylovorans, Bacillus thuringiensis, Bacillus weihenstephanensis, Bacteroidales bacterium ph8, Bacteroidales genomosp. P1, Bacteroidales genomosp. P2 oral clone MB1_G13, Bacteroidales genomosp. P3 oral clone MB1_G34, Bacteroidales genomosp. P4 oral clone MB2_G17, Bacteroidales genomosp. P5 oral clone MB2_P04, Bacteroidales genomosp. P6 oral clone MB3_C9, Bacteroidales genomosp. P7 oral clone MB3_P19, Bacteroidales genomosp. P8 oral clone MB4_G15, Bacteroides acidifaciens, Bacteroides barnesiae, Bacteroides caccae, Bacteroides cellulosilyticus, Bacteroides clarus, Bacteroides coagulans, Bacteroides coprocola, Bacteroides coprophilus, Bacteroides dorei, Bacteroides eggerthii, Bacteroides faecis, Bacteroides finegoldii, Bacteroides fluxus, Bacteroides fragilis, Bacteroides galacturonicus, Bacteroides helcogenes, Bacteroides heparinolyticus, Bacteroides intestinalis, Bacteroides massiliensis, Bacteroides nordii, Bacteroides oleiciplenus, Bacteroides ovatus, Bacteroides pectinophilus, Bacteroides plebeius, Bacteroides pyogenes, Bacteroides salanitronis, Bacteroides salyersiae, Bacteroides sp. 1_1_14, Bacteroides sp. 1_1_30, Bacteroides sp. 1_1_6, Bacteroides sp. 2_1_22, Bacteroides sp. 2_1_56FAA, Bacteroides sp. 2_2_4, Bacteroides sp. 203, Bacteroides sp. 3_1_19, Bacteroides sp. 3_1_23, Bacteroides sp. 3_1_33FAA, Bacteroides sp. 3_1_40A, Bacteroides sp. 3_2_5, Bacteroides sp. 3155, Bacteroides sp. 31SF15, Bacteroides sp. 31SF18, Bacteroides sp. 35AE31, Bacteroides sp. 35AE37, Bacteroides sp. 35BE34, Bacteroides sp. 35BE35, Bacteroides sp. 4_1_36, Bacteroides sp. 4_3_47FAA, Bacteroides sp. 9_1_42FAA, Bacteroides sp. AR20, Bacteroides sp. AR29, Bacteroides sp. B2, Bacteroides sp. D1, Bacteroides sp. D2, Bacteroides sp. D20, Bacteroides sp. D22, Bacteroides sp. F_4, Bacteroides sp. NB_8, Bacteroides sp. WH2, Bacteroides sp. XB12B, Bacteroides sp. XB44A, Bacteroides stercoris, Bacteroides thetaiotaomicron, Bacteroides uniformis, Bacteroides ureolyticus, Bacteroides vulgatus, Bacteroides xylanisolvens, Bacteroidetes bacterium oral taxon D27, Bacteroidetes bacterium oral taxon F31, Bacteroidetes bacterium oral taxon F44, Barnesiella intestinihominis, Barnesiella viscericola, Bartonella bacilliformis, Bartonella grahamii, Bartonella henselae, Bartonella quintana, Bartonella tamiae, Bartonella washoensis, Bdellovibrio sp. MPA, Bifidobacteriaceae genomosp. C1, Bifidobacterium adolescentis, Bifidobacterium angulatum, Bifidobacterium animalis, Bifidobacterium bifidum, Bifidobacterium breve, Bifidobacterium catenulatum, Bifidobacterium dentium, Bifidobacterium gallicum, Bifidobacterium infantis, Bifidobacterium kashiwanohense, Bifidobacterium longum, Bifidobacterium pseudocatenulatum, Bifidobacterium pseudolongum, Bifidobacterium scardovii, Bifidobacterium sp. HM2, Bifidobacterium sp. HMLN12, Bifidobacterium sp. M45, Bifidobacterium sp. MSX5B, Bifidobacterium sp. TM_7, Bifidobacterium thermophilum, Bifidobacterium urinalis, Bilophila wadsworthia, Bisgaard Taxon, Bisgaard Taxon, Bisgaard Taxon, Bisgaard Taxon, Blastomonas natatoria, Blautia coccoides, Blautia glucerasea, Blautia glucerasei, Blautia hansenii, Blautia hydrogenotrophica, Blautia luti, Blautia producta, Blautia schinkii, Blautia sp. M25, Blautia stercoris, Blautia wexlerae, Bordetella bronchiseptica, Bordetella holmesii, Bordetella parapertussis, Bordetella pertussis, Borrelia afzelii, Borrelia burgdorferi, Borrelia crocidurae, Borrelia duttonii, Borrelia garinii, Borrelia hermsii, Borrelia hispanica, Borrelia persica, Borrelia recurrentis, Borrelia sp. NE49, Borrelia spielmanii, Borrelia turicatae, Borrelia valaisiana, Brachybacterium alimentarium, Brachybacterium conglomeratum, Brachybacterium tyrofermentans, Brachyspira aalborgi, Brachyspira pilosicoli, Brachyspira sp. HIS3, Brachyspira sp. HIS4, Brachyspira sp. HIS5, Brevibacillus agri, Brevibacillus brevis, Brevibacillus centrosporus, Brevibacillus choshinensis, Brevibacillus invocatus, Brevibacillus laterosporus, Brevibacillus parabrevis, Brevibacillus reuszeri, Brevibacillus sp. phR, Brevibacillus thermoruber, Brevibacterium aurantiacum, Brevibacterium casei, Brevibacterium epidermidis, Brevibacterium frigoritolerans, Brevibacterium linens, Brevibacterium mcbrellneri, Brevibacterium paucivorans, Brevibacterium sanguinis, Brevibacterium sp. H15, Brevibacterium sp. JC43, Brevundimonas subvibrioides, Brucella abortus, Brucella canis, Brucella ceti, Brucella melitensis, Brucella microti, Brucella ovis, Brucella sp. 83_13, Brucella sp. BO1, Brucella suis, Bryantella formatexigens, Buchnera aphidicola, Bulleidia extructa, Burkholderia ambifaria, Burkholderia cenocepacia, Burkholderia cepacia, Burkholderia mallei, Burkholderia multivorans, Burkholderia oklahomensis, Burkholderia pseudomallei, Burkholderia rhizoxinica, Burkholderia sp. 383, Burkholderia xenovorans, Burkholderiales bacterium 1_1_47, Butyricicoccus pullicaecorum, Butyricimonas virosa, Butyrivibrio crossotus, Butyrivibrio fibrisolvens, Caldimonas manganoxidans, Caminicella sporogenes, Campylobacter coli, Campylobacter concisus, Campylobacter curvus, Campylobacter fetus, Campylobacter gracilis, Campylobacter hominis, Campylobacter jejuni, Campylobacter lari, Campylobacter rectus, Campylobacter showae, Campylobacter sp. FOBRC14, Campylobacter sp. FOBRC15, Campylobacter sp. oral clone BB120, Campylobacter sputorum, Campylobacter upsaliensis, Candidatus arthromitus sp. SFB_mouse_Yit, Candidatus sulcia muelleri, Capnocytophaga canimorsus, Capnocytophaga genomosp. C1, Capnocytophaga gingivalis, Capnocytophaga granulosa, Capnocytophaga ochracea, Capnocytophaga sp. GEJ8, Capnocytophaga sp. oral clone AH015, Capnocytophaga sp. oral clone ASCH05, Capnocytophaga sp. oral clone ID062, Capnocytophaga sp. oral strain A47ROY, Capnocytophaga sp. oral strain S3, Capnocytophaga sp. oral taxon 338, Capnocytophaga sp. Sib, Capnocytophaga sputigena, Cardiobacterium hominis, Cardiobacterium valvarum, Carnobacterium divergens, Carnobacterium maltaromaticum, Catabacter hongkongensis, Catenibacterium mitsuokai, Catonella genomosp. P1 oral clone MB5_P12, Catonella morbi, Catonella sp. oral clone FL037, Cedecea davisae, Cellulosimicrobiumfunkei, Cetobacterium somerae, Chlamydia muridarum, Chlamydia psittaci, Chlamydia trachomatis, Chlamydiales bacterium NS11, Chlamydiales bacterium NS13, Chlamydiales bacterium NS16, Chlamydophila pecorum, Chlamydophila pneumoniae, Chlamydophila psittaci, Chloroflexi genomosp. P1, Christensenella minuta, Chromobacterium violaceum, Chryseobacterium anthropi, Chryseobacterium gleum, Chryseobacterium hominis, Citrobacter amalonaticus, Citrobacter braakii, Citrobacter farmeri, Citrobacter freundii, Citrobacter gillenii, Citrobacter koseri, Citrobacter murliniae, Citrobacter rodentium, Citrobacter sedlakii, Citrobacter sp. 30_2, Citrobacter sp. KMSI_3, Citrobacter werkmanii, Citrobacter youngae, Cloacibacillus evryensis, Clostridiaceae bacterium END_2, Clostridiaceae bacterium JC13, Clostridiales bacterium 1_7_47FAA, Clostridiales bacterium 9400853, Clostridiales bacterium 9403326, Clostridiales bacterium oral clone P4PA_66 P1, Clostridiales bacterium oral taxon 093, Clostridiales bacterium oral taxon F32, Clostridiales bacterium ph2, Clostridiales bacterium SY8519, Clostridiales genomosp. BVAB3, Clostridiales sp. SM4_1, Clostridiales sp. SS3_4, Clostridiales sp. SSC_2, Clostridium acetobutylicum, Clostridium aerotolerans, Clostridium aldenense, Clostridium aldrichii, Clostridium algidicarnis, Clostridium algidixylanolyticum, Clostridium aminovalericum, Clostridium amygdalinum, Clostridium argentinense, Clostridium asparagiforme, Clostridium baratii, Clostridium bartlettii, Clostridium beijerinckii, Clostridium bifermentans, Clostridium bolteae, Clostridium botulinum, Clostridium butyricum, Clostridium cadaveris, Clostridium carboxidivorans, Clostridium carnis, Clostridium celatum, Clostridium celerecrescens, Clostridium cellulosi, Clostridium chauvoei, Clostridium citroniae, Clostridium clariflavum, Clostridium clostridiiformes, Clostridium clostridioforme, Clostridium coccoides, Clostridium cochlearium, Clostridium cocleatum, Clostridium colicanis, Clostridium colinum, Clostridium difficile, Clostridium disporicum, Clostridium estertheticum, Clostridium fallax, Clostridium favososporum, Clostridium felsineum, Clostridium frigidicarnis, Clostridium gasigenes, Clostridium ghonii, Clostridium glycolicum, Clostridium glycyrrhizinilyticum, Clostridium haemolyticum, Clostridium hathewayi, Clostridium hiranonis, Clostridium histolyticum, Clostridium hylemonae, Clostridium indolis, Clostridium innocuum, Clostridium irregulare, Clostridium isatidis, Clostridium kluyveri, Clostridium lactatifermentans, Clostridium lavalense, Clostridium leptum, Clostridium limosum, Clostridium magnum, Clostridium malenominatum, Clostridium mayombei, Clostridium methylpentosum, Clostridium nexile, Clostridium novyi, Clostridium orbiscindens, Clostridium oroticum, Clostridium paraputrificum, Clostridium perfringens, Clostridium phytofermentans, Clostridium piliforme, Clostridium putrefaciens, Clostridium quinii, Clostridium ramosum, Clostridium rectum, Clostridium saccharogumia, Clostridium saccharolyticum, Clostridium sardiniense, Clostridium sartagoforme, Clostridium scindens, Clostridium septicum, Clostridium sordellii, Clostridium sp. 7_2_43FAA, Clostridium sp. D5, Clostridium sp. HGF2, Clostridium sp. HPB_46, Clostridium sp. JC122, Clostridium sp. L2_50, Clostridium sp. LMG 16094, Clostridium sp. M62_1, Clostridium sp. MLG055, Clostridium sp. MT4 E, Clostridium sp. NMBH1_1, Clostridium sp. NML 04A032, Clostridium sp. SS2_1, Clostridium sp. SY8519, Clostridium sp. TM_40, Clostridium sp. YIT 12069, Clostridium sp. YIT 12070, Clostridium sphenoides, Clostridium spiroforme, Clostridium sporogenes, Clostridium sporosphaeroides, Clostridium stercorarium, Clostridium sticklandii, Clostridium straminisolvens, Clostridium subterminale, Clostridium sulfidigenes, Clostridium symbiosum, Clostridium tertium, Clostridium tetani, Clostridium thermocellum, Clostridium tyrobutyricum, Clostridium viride, Clostridium xylanolyticum, Collinsella aerofaciens, Collinsella intestinalis, Collinsella stercoris, Collinsella tanakaei, Comamonadaceae bacterium NML000135, Comamonadaceae bacterium NML790751, Comamonadaceae bacterium NML910035, Comamonadaceae bacterium NML910036, Comamonadaceae bacterium oral taxon F47, Comamonas sp. NSP5, Conchiformibius kuhniae, Coprobacillus cateniformis, Coprobacillus sp. 29_1, Coprobacillus sp. D7, Coprococcus catus, Coprococcus comes, Coprococcus eutactus, Coprococcus sp. ART55_1, Coriobacteriaceae bacterium BV3Ac1, Coriobacteriaceae bacterium JC110, Coriobacteriaceae bacterium ph1, Corynebacterium accolens, Corynebacterium ammoniagenes, Corynebacterium amycolatum, Corynebacterium appendicis, Corynebacterium argentoratense, Corynebacterium atypicum, Corynebacterium aurimucosum, Corynebacterium bovis, Corynebacterium canis, Corynebacterium casei, Corynebacterium confusum, Corynebacterium coyleae, Corynebacterium diphtheriae, Corynebacterium durum, Corynebacterium efficiens, Corynebacterium falsenii, Corynebacterium flavescens, Corynebacterium genitalium, Corynebacterium glaucum, Corynebacterium glucuronolyticum, Corynebacterium glutamicum, Corynebacterium hansenii, Corynebacterium imitans, Corynebacterium jeikeium, Corynebacterium kroppenstedtii, Corynebacterium lipophiloflavum, Corynebacterium macginleyi, Corynebacterium mastitidis, Corynebacterium matruchotii, Corynebacterium minutissimum, Corynebacterium mucifaciens, Corynebacterium propinquum, Corynebacterium pseudodiphtheriticum, Corynebacterium pseudogenitalium, Corynebacterium pseudotuberculosis, Corynebacterium pyruviciproducens, Corynebacterium renale, Corynebacterium resistens, Corynebacterium riegelii, Corynebacterium simulans, Corynebacterium singulare, Corynebacterium sp. 1 ex sheep, Corynebacterium sp. L_2012475, Corynebacterium sp. NML 93_0481, Corynebacterium sp. NML 97_0186, Corynebacterium sp. NML 99_0018, Corynebacterium striatum, Corynebacterium sundsvallense, Corynebacterium tuberculostearicum, Corynebacterium tuscaniae, Corynebacterium ulcerans, Corynebacterium urealyticum, Corynebacterium ureicelerivorans, Corynebacterium variabile, Corynebacterium xerosis, Coxiella burnetii, Cronobacter malonaticus, Cronobacter sakazakii, Cronobacter turicensis, Cryptobacterium curtum, Cupriavidus metallidurans, Cytophaga xylanolytica, Deferribacteres sp. oral clone JV001, Deferribacteres sp. oral clone JV006, Deferribacteres sp. oral clone JV023, Deinococcus radiodurans, Deinococcus sp. R_43890, Delftia acidovorans, Dermabacter hominis, Dermacoccus sp. Ellin185, Desmospora activa, Desmospora sp. 8437, Desulfitobacterium frappieri, Desulfitobacterium hafniense, Desulfobulbus sp. oral clone CH031, Desulfotomaculum nigrificans, Desulfovibrio desulfuricans, Desulfovibrio fairfieldensis, Desulfovibrio piger, Desulfovibrio sp. 3_1_syn3, Desulfovibrio vulgaris, Dialister invisus, Dialister micraerophilus, Dialister microaerophilus, Dialister pneumosintes, Dialister propionicifaciens, Dialister sp. oral taxon 502, Dialister succinatiphilus, Dietzia natronolimnaea, Dietzia sp. BBDP51, Dietzia sp. CA149, Dietzia timorensis, Dorea formicigenerans, Dorea longicatena, Dysgonomonas gadei, Dysgonomonas mossii, Edwardsiella tarda, Eggerthella lenta, Eggerthella sinensis, Eggerthella sp. 1_3_56FAA, Eggerthella sp. HGA1, Eggerthella sp. YY7918, Ehrlichia chaffeensis, Eikenella corrodens, Enhydrobacter aerosaccus, Enterobacter aerogenes, Enterobacter asburiae, Enterobacter cancerogenus, Enterobacter cloacae, Enterobacter cowanii, Enterobacter hormaechei, Enterobacter sp. 247BMC, Enterobacter sp. 638, Enterobacter sp. JC163, Enterobacter sp. SCSS, Enterobacter sp. TSE38, Enterobacteriaceae bacterium 9_2_54FAA, Enterobacteriaceae bacterium CF01Ent_1, Enterobacteriaceae bacterium Smarlab 3302238, Enterococcus avium, Enterococcus caccae, Enterococcus casseliflavus, Enterococcus durans, Enterococcus faecalis, Enterococcus faecium, Enterococcus gallinarum, Enterococcus gilvus, Enterococcus hawaiiensis, Enterococcus hirae, Enterococcus italicus, Enterococcus mundtii, Enterococcus raffinosus, Enterococcus sp. BV2CASA2, Enterococcus sp. CCR1_16620, Enterococcus sp. F95, Enterococcus sp. RfL6, Enterococcus thailandicus, Eremococcus coleocola, Erysipelothrix inopinata, Erysipelothrix rhusiopathiae, Erysipelothrix tonsillarum, Erysipelotrichaceae bacterium 3_1_53, Erysipelotrichaceae bacterium 5_2_54FAA, Escherichia albertii, Escherichia coli, Escherichia fergusonii, Escherichia hermannii, Escherichia sp. 1_1_43, Escherichia sp. 4_1_40B, Escherichia sp. B4, Escherichia vulneris, Ethanoligenens harbinense, Eubacteriaceae bacterium P4P_50 P4, Eubacterium barkeri, Eubacterium biforme, Eubacterium brachy, Eubacterium budayi, Eubacterium callanderi, Eubacterium cellulosolvens, Eubacterium contortum, Eubacterium coprostanoligenes, Eubacterium cylindroides, Eubacterium desmolans, Eubacterium dolichum, Eubacterium eligens, Eubacterium fissicatena, Eubacterium hadrum, Eubacterium hallii, Eubacterium infirmum, Eubacterium limosum, Eubacterium moniliforme, Eubacterium multiforme, Eubacterium nitritogenes, Eubacterium nodatum, Eubacterium ramulus, Eubacterium rectale, Eubacterium ruminantium, Eubacterium saburreum, Eubacterium saphenum, Eubacterium siraeum, Eubacterium sp. 3_1_31, Eubacterium sp. AS15b, Eubacterium sp. OBRC9, Eubacterium sp. oral clone GI038, Eubacterium sp. oral clone IR009, Eubacterium sp. oral clone JH012, Eubacterium sp. oral clone JI012, Eubacterium sp. oral clone JN088, Eubacterium sp. oral clone JS001, Eubacterium sp. oral clone OH3A, Eubacterium sp. WAL 14571, Eubacterium tenue, Eubacterium tortuosum, Eubacterium ventriosum, Eubacterium xylanophilum, Eubacterium yurii, Ewingella americana, Exiguobacterium acetylicum, Facklamia hominis, Faecalibacterium prausnitzii, Filifactor alocis, Filifactor villosus, Finegoldia magna, Flavobacteriaceae genomosp. C1, Flavobacterium sp. NF2_1, Flavonifractor plautii, Flexispira rappini, Flexistipes sinusarabici, Francisella novicida, Francisella philomiragia, Francisella tularensis, Fulvimonas sp. NML 060897, Fusobacterium canifelinum, Fusobacterium genomosp. C1, Fusobacterium genomosp. C2, Fusobacterium gonidiaformans, Fusobacterium mortiferum, Fusobacterium naviforme, Fusobacterium necrogenes, Fusobacterium necrophorum, Fusobacterium nucleatum, Fusobacterium periodonticum, Fusobacterium russii, Fusobacterium sp. 1_1_41FAA, Fusobacterium sp. 11_3_2, Fusobacterium sp. 12_1B, Fusobacterium sp. 2_1_31, Fusobacterium sp. 3_1_27, Fusobacterium sp. 3_1_33, Fusobacterium sp. 3_1_36A2, Fusobacterium sp. 3_1_5R, Fusobacterium sp. AC18, Fusobacterium sp. ACB2, Fusobacterium sp. AS2, Fusobacterium sp. CM1, Fusobacterium sp. CM21, Fusobacterium sp. CM22, Fusobacterium sp. D12, Fusobacterium sp. oral clone ASCF06, Fusobacterium sp. oral clone ASCF11, Fusobacterium ulcerans, Fusobacterium varium, Gardnerella vaginalis, Gemella haemolysans, Gemella morbillorum, Gemella morbillorum, Gemella sanguinis, Gemella sp. oral clone ASCE02, Gemella sp. oral clone ASCF04, Gemella sp. oral clone ASCF12, Gemella sp. WAL 1945J, Gemmiger formicilis, Geobacillus kaustophilus, Geobacillus sp. E263, Geobacillus sp. WCH70, Geobacillus stearothermophilus, Geobacillus thermocatenulatus, Geobacillus thermodenitrificans, Geobacillus thermoglucosidasius, Geobacillus thermoleovorans, Geobacter bemidjiensis, Gloeobacter violaceus, Gluconacetobacter azotocaptans, Gluconacetobacter diazotrophicus, Gluconacetobacter entanii, Gluconacetobacter europaeus, Gluconacetobacter hansenii, Gluconacetobacter johannae, Gluconacetobacter oboediens, Gluconacetobacter xylinus, Gordonia bronchialis, Gordonia polyisoprenivorans, Gordonia sp. KTR9, Gordonia sputi, Gordonia terrae, Gordonibacter pamelaeae, Gordonibacter pamelaeae, Gracilibacter thermotolerans, Gramella forsetii, Granulicatella adiacens, Granulicatella elegans, Granulicatella paradiacens, Granulicatella sp. M658_99_3, Granulicatella sp. oral clone ASC02, Granulicatella sp. oral clone ASCA05, Granulicatella sp. oral clone ASCB09, Granulicatella sp. oral clone ASCGO5, Grimontia hollisae, Haematobacter sp. BC14248, Haemophilus aegyptius, Haemophilus ducreyi, Haemophilus genomosp. P2 oral clone MB3_C24, Haemophilus genomosp. P3 oral clone MB3_C38, Haemophilus haemolyticus, Haemophilus influenzae, Haemophilus parahaemolyticus, Haemophilus parainfluenzae, Haemophilus paraphrophaemolyticus, Haemophilus parasuis, Haemophilus somnus, Haemophilus sp. 70334, Haemophilus sp. HK445, Haemophilus sp. oral clone ASCA07, Haemophilus sp. oral clone ASCG06, Haemophilus sp. oral clone BJ021, Haemophilus sp. oral clone BJ095, Haemophilus sp. oral clone JM053, Haemophilus sp. oral taxon 851, Haemophilus sputorum, Hafnia alvei, Halomonas elongata, Halomonas johnsoniae, Halorubrum lipolyticum, Helicobacter bilis, Helicobacter canadensis, Helicobacter cinaedi, Helicobacter pullorum, Helicobacter pylori, Helicobacter sp. None, Helicobacter winghamensis, Heliobacterium modesticaldum, Herbaspirillum seropedicae, Herbaspirillum sp. JC206, Histophilus somni, Holdemania filiformis, Hydrogenoanaerobacterium saccharovorans, Hyperthermus butylicus, Hyphomicrobium sulfonivorans, Hyphomonas neptunium, Ignatzschineria indica, Ignatzschineria sp. NML 95_0260, Ignicoccus islandicus, Inquilinus limosus, Janibacter limosus, Janibacter melonis, Janthinobacterium sp. SY12, Johnsonella ignava, Jonquetella anthropi, Kerstersia gyiorum, Kingella denitrificans, Kingella genomosp. P1 oral cone MB2_C20, Kingella kingae, Kingella oralis, Kingella sp. oral clone ID059, Klebsiella oxytoca, Klebsiella pneumoniae, Klebsiella sp. AS10, Klebsiella sp. Co9935, Klebsiella sp. enrichment culture clone SRC_DSD25, Klebsiella sp. OBRC7, Klebsiella sp. SP_BA, Klebsiella sp. SRC_DSD1, Klebsiella sp. SRC_DSD11, Klebsiella sp. SRC_DSD12, Klebsiella sp. SRC_DSD15, Klebsiella sp. SRC_DSD2, Klebsiella sp. SRC_DSD6, Klebsiella variicola, Kluyvera ascorbata, Kluyvera cryocrescens, Kocuria marina, Kocuria palustris, Kocuria rhizophila, Kocuria rosea, Kocuria varians, Lachnobacterium bovis, Lachnospira multipara, Lachnospira pectinoschiza, Lachnospiraceae bacterium 1_1_57FAA, Lachnospiraceae bacterium 1_4_56FAA, Lachnospiraceae bacterium 2_1_46FAA, Lachnospiraceae bacterium 2_1_58FAA, Lachnospiraceae bacterium 3_1_57FAA_CT1, Lachnospiraceae bacterium 4_1_37FAA, Lachnospiraceae bacterium 5_1_57FAA, Lachnospiraceae bacterium 5_1_63FAA, Lachnospiraceae bacterium 6_1_63FAA, Lachnospiraceae bacterium 8_1_57FAA, Lachnospiraceae bacterium 9_1_43BFAA, Lachnospiraceae bacterium A4, Lachnospiraceae bacterium DJF VP30, Lachnospiraceae bacterium ICM62, Lachnospiraceae bacterium MSX33, Lachnospiraceae bacterium oral taxon 107, Lachnospiraceae bacterium oral taxon F15, Lachnospiraceae genomosp. C1, Lactobacillus acidipiscis, Lactobacillus acidophilus, Lactobacillus alimentarius, Lactobacillus amylolyticus, Lactobacillus amylovorus, Lactobacillus antri, Lactobacillus brevis, Lactobacillus buchneri, Lactobacillus casei, Lactobacillus catenaformis, Lactobacillus coleohominis, Lactobacillus coryniformis, Lactobacillus crispatus, Lactobacillus curvatus, Lactobacillus delbrueckii, Lactobacillus dextrinicus, Lactobacillus farciminis, Lactobacillus fermentum, Lactobacillus gasseri, Lactobacillus gastricus, Lactobacillus genomosp. C1, Lactobacillus genomosp. C2, Lactobacillus helveticus, Lactobacillus hilgardii, Lactobacillus hominis, Lactobacillus iners, Lactobacillus jensenii, Lactobacillus johnsonii, Lactobacillus kalixensis, Lactobacillus kefiranofaciens, Lactobacillus kefiri, Lactobacillus kimchii, Lactobacillus leichmannii, Lactobacillus mucosae, Lactobacillus murinus, Lactobacillus nodensis, Lactobacillus oeni, Lactobacillus oris, Lactobacillus parabrevis, Lactobacillus parabuchneri, Lactobacillus paracasei, Lactobacillus parakefiri, Lactobacillus pentosus, Lactobacillus perolens, Lactobacillus plantarum, Lactobacillus pontis, Lactobacillus reuteri, Lactobacillus rhamnosus, Lactobacillus rogosae, Lactobacillus ruminis, Lactobacillus sakei, Lactobacillus salivarius, Lactobacillus saniviri, Lactobacillus senioris, Lactobacillus sp. 66c, Lactobacillus sp. BT6, Lactobacillus sp. KLDS 1.0701, Lactobacillus sp. KLDS 1.0702, Lactobacillus sp. KLDS 1.0703, Lactobacillus sp. KLDS 1.0704, Lactobacillus sp. KLDS 1.0705, Lactobacillus sp. KLDS 1.0707, Lactobacillus sp. KLDS 1.0709, Lactobacillus sp. KLDS 1.0711, Lactobacillus sp. KLDS 1.0712, Lactobacillus sp. KLDS 1.0713, Lactobacillus sp. KLDS 1.0716, Lactobacillus sp. KLDS 1.0718, Lactobacillus sp. KLDS 1.0719, Lactobacillus sp. oral clone HT002, Lactobacillus sp. oral clone HT070, Lactobacillus sp. oral taxon 052, Lactobacillus tucceti, Lactobacillus ultunensis, Lactobacillus vaginalis, Lactobacillus vini, Lactobacillus vitulinus, Lactobacillus zeae, Lactococcus garvieae, Lactococcus lactis, Lactococcus raffinolactis, Lactonifactor longoviformis, Laribacter hongkongensis, Lautropia mirabilis, Lautropia sp. oral clone AP009, Legionella hackeliae, Legionella longbeachae, Legionella pneumophila, Legionella sp. D3923, Legionella sp. D4088, Legionella sp. H63, Legionella sp. NML 93L054, Legionella steelei, Leminorella grimontii, Leminorella richardii, Leptospira borgpetersenii, Leptospira broomii, Leptospira interrogans, Leptospira licerasiae, Leptotrichia buccalis, Leptotrichia genomosp. C1, Leptotrichia goodfellowii, Leptotrichia hofstadii, Leptotrichia shahii, Leptotrichia sp. neutropenicPatient, Leptotrichia sp. oral clone GT018, Leptotrichia sp. oral clone GT020, Leptotrichia sp. oral clone HE012, Leptotrichia sp. oral clone IK040, Leptotrichia sp. oral clone P2PB_51 P1, Leptotrichia sp. oral taxon 223, Leuconostoc carnosum, Leuconostoc citreum, Leuconostoc gasicomitatum, Leuconostoc inhae, Leuconostoc kimchii, Leuconostoc lactis, Leuconostoc mesenteroides, Leuconostoc pseudomesenteroides, Listeria grayi, Listeria innocua, Listeria ivanovii, Listeria monocytogenes, Listeria welshimeri, Luteococcus sanguinis, Lutispora thermophila, Lysinibacillus fusiformis, Lysinibacillus sphaericus, Macrococcus caseolyticus, Mannheimia haemolytica, Marvinbryantia formatexigens, Massilia sp. CCUG 43427A, Megamonas funiformis, Megamonas hypermegale, Megasphaera elsdenii, Megasphaera genomosp. C1, Megasphaera genomosp. type_1, Megasphaera micronuciformis, Megasphaera sp. BLPYG_07, Megasphaera sp. UPII 199_6, Metallosphaera sedula, Methanobacterium formicicum, Methanobrevibacter acididurans, Methanobrevibacter arboriphilus, Methanobrevibacter curvatus, Methanobrevibacter cuticularis, Methanobrevibacter filiformis, Methanobrevibacter gottschalkii, Methanobrevibacter millerae, Methanobrevibacter olleyae, Methanobrevibacter oralis, Methanobrevibacter ruminantium, Methanobrevibacter smithii, Methanobrevibacter thaueri, Methanobrevibacter woesei, Methanobrevibacter wolinii, Methanosphaera stadtmanae, Methylobacterium extorquens, Methylobacterium podarium, Methylobacterium radiotolerans, Methylobacterium sp. 1sub, Methylobacterium sp. MM4, Methylocella silvestris, Methylophilus sp. ECd5, Microbacterium chocolatum, Microbacteriumflavescens, Microbacterium gubbeenense, Microbacterium lacticum, Microbacterium oleivorans, Microbacterium oxydans, Microbacterium paraoxydans, Microbacterium phyllosphaerae, Microbacterium schleiferi, Microbacterium sp. 768, Microbacterium sp. oral strain C24KA, Microbacterium testaceum, Micrococcus antarcticus, Micrococcus luteus, Micrococcus lylae, Micrococcus sp. 185, Microcystis aeruginosa, Mitsuokella jalaludinii, Mitsuokella multacida, Mitsuokella sp. oral taxon 521, Mitsuokella sp. oral taxon G68, Mobiluncus curtisii, Mobiluncus mulieris, Moellerella wisconsensis, Mogibacterium diversum, Mogibacterium neglectum, Mogibacterium pumilum, Mogibacterium timidum, Mollicutes bacterium pACH93, Moorella thermoacetica, Moraxella catarrhalis, Moraxella lincolnii, Moraxella osloensis, Moraxella sp. 16285, Moraxella sp. GM2, Morganella morganii, Morganella sp. JB_T16, Morococcus cerebrosus, Moryella indoligenes, Mycobacterium abscessus, Mycobacterium africanum, Mycobacterium alsiensis, Mycobacterium avium, Mycobacterium chelonae, Mycobacterium colombiense, Mycobacterium elephantis, Mycobacterium gordonae, Mycobacterium intracellulare, Mycobacterium kansasii, Mycobacterium lacus, Mycobacterium leprae, Mycobacterium lepromatosis, Mycobacterium mageritense, Mycobacterium mantenii, Mycobacterium marinum, Mycobacterium microti, Mycobacterium neoaurum, Mycobacterium parascrofulaceum, Mycobacterium paraterrae, Mycobacterium phlei, Mycobacterium seoulense, Mycobacterium smegmatis, Mycobacterium sp. 1761, Mycobacterium sp. 1776, Mycobacterium sp. 1781, Mycobacterium sp. 1791, Mycobacterium sp. 1797, Mycobacterium sp. AQ1GA4, Mycobacterium sp. B10_07.09.0206, Mycobacterium sp. GN_10546, Mycobacterium sp. GN_10827, Mycobacterium sp. GN_11124, Mycobacterium sp. GN_9188, Mycobacterium sp. GR_2007_210, Mycobacterium sp. HES, Mycobacterium sp. NLA001000736, Mycobacterium sp. W, Mycobacterium tuberculosis, Mycobacterium ulcerans, Mycobacterium vulneris, Mycoplasma agalactiae, Mycoplasma amphoriforme, Mycoplasma arthritidis, Mycoplasma bovoculi, Mycoplasma faucium, Mycoplasma fermentans, Mycoplasma flocculare, Mycoplasma genitalium, Mycoplasma hominis, Mycoplasma orale, Mycoplasma ovipneumoniae, Mycoplasma penetrans, Mycoplasma pneumoniae, Mycoplasma putrefaciens, Mycoplasma salivarium, Mycoplasmataceae genomosp. P1 oral clone MB1_G23, Myroides odoratimimus, Myroides sp. MY15, Neisseria bacilliformis, Neisseria cinerea, Neisseria elongata, Neisseria flavescens, Neisseria genomosp. P2 oral clone MB5_P15, Neisseria gonorrhoeae, Neisseria lactamica, Neisseria macacae, Neisseria meningitidis, Neisseria mucosa, Neisseria pharyngis, Neisseria polysaccharea, Neisseria sicca, Neisseria sp. KEM232, Neisseria sp. oral clone AP132, Neisseria sp. oral clone JC012, Neisseria sp. oral strain B33KA, Neisseria sp. oral taxon 014, Neisseria sp. SMC_A9199, Neisseria sp. TM10_1, Neisseria subflava, Neorickettsia risticii, Neorickettsia sennetsu, Nocardia brasiliensis, Nocardia cyriacigeorgica, Nocardia farcinica, Nocardia puris, Nocardia sp. 01_Je_025, Nocardiopsis dassonvillei, Novosphingobium aromaticivorans, Oceanobacillus caeni, Oceanobacillus sp. Ndiop, Ochrobactrum anthropi, Ochrobactrum intermedium, Ochrobactrum pseudintermedium, Odoribacter laneus, Odoribacter splanchnicus, Okadaella gastrococcus, Oligella ureolytica, Oligella urethralis, Olsenella genomosp. C1, Olsenella profusa, Olsenella sp. F0004, Olsenella sp. oral taxon 809, Olsenella uli, Opitutus terrae, Oribacterium sinus, Oribacterium sp. ACB1, Oribacterium sp. ACB7, Oribacterium sp. CM12, Oribacterium sp. ICM51, Oribacterium sp. OBRC12, Oribacterium sp. oral taxon 078, Oribacterium sp. oral taxon 102, Oribacterium sp. oral taxon 108, Orientia tsutsugamushi, Ornithinibacillus bavariensis, Ornithinibacillus sp. 7_10AIA, Oscillibacter sp. G2, Oscillibacter valericigenes, Oscillospira guilliermondii, Oxalobacter formigenes, Paenibacillus barcinonensis, Paenibacillus barengoltzii, Paenibacillus chibensis, Paenibacillus cookii, Paenibacillus durus, Paenibacillus glucanolyticus, Paenibacillus lactis, Paenibacillus lautus, Paenibacillus pabuli, Paenibacillus polymyxa, Paenibacillus popilliae, Paenibacillus sp. CIP 101062, Paenibacillus sp. HGF5, Paenibacillus sp. HGF7, Paenibacillus sp. JC66, Paenibacillus sp. oral taxon F45, Paenibacillus sp. R_27413, Paenibacillus sp. R_27422, Paenibacillus timonensis, Pantoea agglomerans, Pantoea ananatis, Pantoea brenneri, Pantoea citrea, Pantoea conspicua, Pantoea septica, Papillibacter cinnamivorans, Parabacteroides distasonis, Parabacteroides goldsteinii, Parabacteroides gordonii, Parabacteroides johnsonii, Parabacteroides merdae, Parabacteroides sp. D13, Parabacteroides sp. NS31_3, Parachlamydia sp. UWE25, Paracoccus denitrificans, Paracoccus marcusii, Paraprevotella clara, Paraprevotella xylaniphila, Parascardovia denticolens, Parasutterella excrementihominis, Parasutterella secunda, Parvimonas micra, Parvimonas sp. oral taxon 110, Pasteurella bettyae, Pasteurella dagmatis, Pasteurella multocida, Pediococcus acidilactici, Pediococcus pentosaceus, Peptococcus niger, Peptococcus sp. oral clone JM048, Peptococcus sp. oral taxon 167, Peptoniphilus asaccharolyticus, Peptoniphilus duerdenii, Peptoniphilus harei, Peptoniphilus indolicus, Peptoniphilus ivorii, Peptoniphilus lacrimalis, Peptoniphilus sp. gpac007, Peptoniphilus sp. gpac018A, Peptoniphilus sp. gpac077, Peptoniphilus sp. gpac148, Peptoniphilus sp. JC140, Peptoniphilus sp. oral taxon 386, Peptoniphilus sp. oral taxon 836, Peptostreptococcaceae bacterium ph1, Peptostreptococcus anaerobius, Peptostreptococcus micros, Peptostreptococcus sp. 9succ1, Peptostreptococcus sp. oral clone AP24, Peptostreptococcus sp. oral clone FJ023, Peptostreptococcus sp. P4P_31 P3, Peptostreptococcus stomatis, Phascolarctobacteriumfaecium, Phascolarctobacterium sp. YIT 12068, Phascolarctobacterium succinatutens, Phenylobacterium zucineum, Photorhabdus asymbiotica, Pigmentiphaga daeguensis, Planomicrobium koreense, Plesiomonas shigelloides, Porphyromonadaceae bacterium NML 060648, Porphyromonas asaccharolytica, Porphyromonas endodontalis, Porphyromonas gingivalis, Porphyromonas levii, Porphyromonas macacae, Porphyromonas somerae, Porphyromonas sp. oral clone BB134, Porphyromonas sp. oral clone F016, Porphyromonas sp. oral clone P2PB_52 P1, Porphyromonas sp. oral clone P4_GB_100 P2, Porphyromonas sp. UQD 301, Porphyromonas uenonis, Prevotella albensis, Prevotella amnii, Prevotella bergensis, Prevotella bivia, Prevotella brevis, Prevotella buccae, Prevotella buccalis, Prevotella copri, Prevotella corporis, Prevotella dentalis, Prevotella denticola, Prevotella disiens, Prevotella genomosp. C1, Prevotella genomosp. C2, Prevotella genomosp. P7 oral clone MB2_P31, Prevotella genomosp. P8 oral clone MB3_P13, Prevotella genomosp. P9 oral clone MB7_G16, Prevotella heparinolytica, Prevotella histicola, Prevotella intermedia, Prevotella loescheii, Prevotella maculosa, Prevotella marshii, Prevotella melaninogenica, Prevotella micans, Prevotella multiformis, Prevotella multisaccharivorax, Prevotella nanceiensis, Prevotella nigrescens, Prevotella oralis, Prevotella oris, Prevotella oulorum, Prevotella pallens, Prevotella ruminicola, Prevotella salivae, Prevotella sp. BI_42, Prevotella sp. CM38, Prevotella sp. ICM1, Prevotella sp. ICM55, Prevotella sp. JCM 6330, Prevotella sp. oral clone AA020, Prevotella sp. oral clone ASCG10, Prevotella sp. oral clone ASCG12, Prevotella sp. oral clone AU069, Prevotella sp. oral clone CY006, Prevotella sp. oral clone DA058, Prevotella sp. oral clone FL019, Prevotella sp. oral clone FU048, Prevotella sp. oral clone FW035, Prevotella sp. oral clone GI030, Prevotella sp. oral clone GI032, Prevotella sp. oral clone GI059, Prevotella sp. oral clone GU027, Prevotella sp. oral clone HF050, Prevotella sp. oral clone ID019, Prevotella sp. oral clone IDR_CEC_0055, Prevotella sp. oral clone IK053, Prevotella sp. oral clone IK062, Prevotella sp. oral clone P4PB_83 P2, Prevotella sp. oral taxon 292, Prevotella sp. oral taxon 299, Prevotella sp. oral taxon 300, Prevotella sp. oral taxon 302, Prevotella sp. oral taxon 310, Prevotella sp. oral taxon 317, Prevotella sp. oral taxon 472, Prevotella sp. oral taxon 781, Prevotella sp. oral taxon 782, Prevotella sp. oral taxon F68, Prevotella sp. oral taxon G60, Prevotella sp. oral taxon G70, Prevotella sp. oral taxon G71, Prevotella sp. SEQ053, Prevotella sp. SEQ065, Prevotella sp. SEQ072, Prevotella sp. SEQ116, Prevotella sp. SG12, Prevotella sp. sp24, Prevotella sp. sp34, Prevotella stercorea, Prevotella tannerae, Prevotella timonensis, Prevotella veroralis, Prevotella jejuni, Prevotella aurantiaca, Prevotella baroniae, Prevotella colorans, Prevotella corporis, Prevotella dentasini, Prevotella enoeca, Prevotella falsenii, Prevotella fusca, Prevotella heparinolytica, Prevotella loescheii, Prevotella multisaccharivorax, Prevotella nanceiensis, Prevotella oryzae, Prevotella paludivivens, Prevotella pleuritidis, Prevotella ruminicola, Prevotella saccharolytica, Prevotella scopos, Prevotella shahii, Prevotella zoogleoformans, Prevotellaceae bacterium P4P_62 P1, Prochlorococcus marinus, Propionibacteriaceae bacterium NML 02_0265, Propionibacterium acidipropionici, Propionibacterium acnes, Propionibacterium avidum, Propionibacterium freudenreichii, Propionibacterium granulosum, Propionibacterium jensenii, Propionibacterium propionicum, Propionibacterium sp. 434_HC2, Propionibacterium sp. H456, Propionibacterium sp. LG, Propionibacterium sp. oral taxon 192, Propionibacterium sp. S555a, Propionibacterium thoenii, Proteus mirabilis, Proteus penneri, Proteus sp. HS7514, Proteus vulgaris, Providencia alcalifaciens, Providencia rettgeri, Providencia rustigianii, Providencia stuartii, Pseudoclavibacter sp. Timone, Pseudoflavonifractor capillosus, Pseudomonas aeruginosa, Pseudomonas fluorescens, Pseudomonas gessardii, Pseudomonas mendocina, Pseudomonas monteilii, Pseudomonas poae, Pseudomonas pseudoalcaligenes, Pseudomonas putida, Pseudomonas sp. 21_26, Pseudomonas sp. G1229, Pseudomonas sp. NP522b, Pseudomonas stutzeri, Pseudomonas tolaasii, Pseudomonas viridiflava, Pseudoramibacter alactolyticus, Psychrobacter arcticus, Psychrobacter cibarius, Psychrobacter cryohalolentis, Psychrobacter faecalis, Psychrobacter nivimaris, Psychrobacter pulmonis, Psychrobacter sp. 13983, Pyramidobacter piscolens, Ralstonia pickettii, Ralstonia sp. 5_7_47FAA, Raoultella ornithinolytica, Raoultella planticola, Raoultella terrigena, Rhodobacter sp. oral taxon C30, Rhodobacter sphaeroides, Rhodococcus corynebacterioides, Rhodococcus equi, Rhodococcus erythropolis, Rhodococcus fascians, Rhodopseudomonas palustris, Rickettsia akari, Rickettsia conorii, Rickettsia prowazekii, Rickettsia rickettsii, Rickettsia slovaca, Rickettsia typhi, Robinsoniella peoriensis, Roseburia cecicola, Roseburia faecalis, Roseburia faecis, Roseburia hominis, Roseburia intestinalis, Roseburia inulinivorans, Roseburia sp. 11SE37, Roseburia sp. 11SE38, Roseiflexus castenholzii, Roseomonas cervicalis, Roseomonas mucosa, Roseomonas sp. NML94_0193, Roseomonas sp. NML97_0121, Roseomonas sp. NML98_0009, Roseomonas sp. NML98_0157, Rothia aeria, Rothia dentocariosa, Rothia mucilaginosa, Rothia nasimurium, Rothia sp. oral taxon 188, Ruminobacter amylophilus, Ruminococcaceae bacterium D16, Ruminococcus albus, Ruminococcus bromii, Ruminococcus callidus, Ruminococcus champanellensis, Ruminococcus flavefaciens, Ruminococcus gnavus, Ruminococcus hansenii, Ruminococcus lactaris, Ruminococcus obeum, Ruminococcus sp. 18P13, Ruminococcus sp. 5_1_39BFAA, Ruminococcus sp. 9SE51, Ruminococcus sp. ID8, Ruminococcus sp. K_1, Ruminococcus torques, Saccharomonospora viridis, Salmonella bongori, Salmonella enterica, Salmonella enterica, Salmonella enterica, Salmonella enterica, Salmonella enterica, Salmonella enterica, Salmonella enterica, Salmonella enterica, Salmonella enterica, Salmonella enterica, Salmonella enterica, Salmonella enterica, Salmonella typhimurium, Salmonella typhimurium, Sarcina ventriculi, Scardovia inopinata, Scardovia wiggsiae, Segniliparus rotundus, Segniliparus rugosus, Selenomonas artemidis, Selenomonas dianae, Selenomonas flueggei, Selenomonas genomosp. C1, Selenomonas genomosp. C2, Selenomonas genomosp. P5, Selenomonas genomosp. P6 oral clone MB3_C41, Selenomonas genomosp. P7 oral clone MB5_C08, Selenomonas genomosp. P8 oral clone MB5_P06, Selenomonas infelix, Selenomonas noxia, Selenomonas ruminantium, Selenomonas sp. FOBRC9, Selenomonas sp. oral clone FT050, Selenomonas sp. oral clone GI064, Selenomonas sp. oral clone GT010, Selenomonas sp. oral clone HU051, Selenomonas sp. oral clone IK004, Selenomonas sp. oral clone IQ048, Selenomonas sp. oral clone JI021, Selenomonas sp. oral clone JS031, Selenomonas sp. oral clone OH4A, Selenomonas sp. oral clone P2PA_80 P4, Selenomonas sp. oral taxon 137, Selenomonas sp. oral taxon 149, Selenomonas sputigena, Serratia fonticola, Serratia liquefaciens, Serratia marcescens, Serratia odorifera, Serratia proteamaculans, Shewanella putrefaciens, Shigella boydii, Shigella dysenteriae, Shigella flexneri, Shigella sonnei, Shuttleworthia satelles, Shuttleworthia sp. MSX8B, Shuttleworthia sp. oral taxon G69, Simonsiella muelleri, Slackia equolifaciens, Slackia exigua, Slackia faecicanis, Slackia heliotrinireducens, Slackia isoflavoniconvertens, Slackia piriformis, Slackia sp. NATTS, Solobacterium moorei, Sphingobacterium faecium, Sphingobacterium mizutaii, Sphingobacterium multivorum, Sphingobacterium spiritivorum, Sphingomonas echinoides, Sphingomonas sp. oral clone FI012, Sphingomonas sp. oral clone FZ016, Sphingomonas sp. oral taxon A09, Sphingomonas sp. oral taxon F71, Sphingopyxis alaskensis, Spiroplasma insolitum, Sporobacter termitidis, Sporolactobacillus inulinus, Sporolactobacillus nakayamae, Sporosarcina newyorkensis, Sporosarcina sp. 2681, Staphylococcaceae bacterium NML 92_0017, Staphylococcus aureus, Staphylococcus auricularis, Staphylococcus capitis, Staphylococcus caprae, Staphylococcus carnosus, Staphylococcus cohnii, Staphylococcus condimenti, Staphylococcus epidermidis, Staphylococcus equorum, Staphylococcus fleurettii, Staphylococcus haemolyticus, Staphylococcus hominis, Staphylococcus lugdunensis, Staphylococcus pasteuri, Staphylococcus pseudintermedius, Staphylococcus saccharolyticus, Staphylococcus saprophyticus, Staphylococcus sciuri, Staphylococcus sp. clone bottae7, Staphylococcus sp. H292, Staphylococcus sp. H780, Staphylococcus succinus, Staphylococcus vitulinus, Staphylococcus warneri, Staphylococcus xylosus, Stenotrophomonas maltophilia, Stenotrophomonas sp. FG_6, Streptobacillus moniliformis, Streptococcus agalactiae, Streptococcus alactolyticus, Streptococcus anginosus, Streptococcus australis, Streptococcus bovis, Streptococcus canis, Streptococcus constellatus, Streptococcus cristatus, Streptococcus downei, Streptococcus dysgalactiae, Streptococcus equi, Streptococcus equinus, Streptococcus gallolyticus, Streptococcus genomosp. C1, Streptococcus genomosp. C2, Streptococcus genomosp. C3, Streptococcus genomosp. C4, Streptococcus genomosp. C5, Streptococcus genomosp. C6, Streptococcus genomosp. C7, Streptococcus genomosp. C8, Streptococcus gordonii, Streptococcus infantarius, Streptococcus infantis, Streptococcus intermedius, Streptococcus lutetiensis, Streptococcus massiliensis, Streptococcus milleri, Streptococcus mitis, Streptococcus mutans, Streptococcus oligofermentans, Streptococcus oralis, Streptococcus parasanguinis, Streptococcus pasteurianus, Streptococcus peroris, Streptococcus pneumoniae, Streptococcus porcinus, Streptococcus pseudopneumoniae, Streptococcus pseudoporcinus, Streptococcus pyogenes, Streptococcus ratti, Streptococcus salivarius, Streptococcus sanguinis, Streptococcus sinensis, Streptococcus sp. 16362, Streptococcus sp. 2_1_36FAA, Streptococcus sp. 228597, Streptococcus sp. 69130, Streptococcus sp. AC15, Streptococcus sp. ACS2, Streptococcus sp. AS20, Streptococcus sp. BS35a, Streptococcus sp. C150, Streptococcus sp. CM6, Streptococcus sp. CM7, Streptococcus sp. ICM10, Streptococcus sp. ICM12, Streptococcus sp. ICM2, Streptococcus sp. ICM4, Streptococcus sp. ICM45, Streptococcus sp. M143, Streptococcus sp. M334, Streptococcus sp. OBRC6, Streptococcus sp. oral clone ASB02, Streptococcus sp. oral clone ASCA03, Streptococcus sp. oral clone ASCA04, Streptococcus sp. oral clone ASCA09, Streptococcus sp. oral clone ASCB04, Streptococcus sp. oral clone ASCB06, Streptococcus sp. oral clone ASCC04, Streptococcus sp. oral clone ASCC05, Streptococcus sp. oral clone ASCC12, Streptococcus sp. oral clone ASCD01, Streptococcus sp. oral clone ASCD09, Streptococcus sp. oral clone ASCD10, Streptococcus sp. oral clone ASCE03, Streptococcus sp. oral clone ASCE04, Streptococcus sp. oral clone ASCE05, Streptococcus sp. oral clone ASCE06, Streptococcus sp. oral clone ASCE09, Streptococcus sp. oral clone ASCE10, Streptococcus sp. oral clone ASCE12, Streptococcus sp. oral clone ASCF05, Streptococcus sp. oral clone ASCF07, Streptococcus sp. oral clone ASCF09, Streptococcus sp. oral clone ASCG04, Streptococcus sp. oral clone BW009, Streptococcus sp. oral clone CH016, Streptococcus sp. oral clone GK051, Streptococcus sp. oral clone GM006, Streptococcus sp. oral clone P2PA_41 P2, Streptococcus sp. oral clone P4PA_30 P4, Streptococcus sp. oral taxon 071, Streptococcus sp. oral taxon G59, Streptococcus sp. oral taxon G62, Streptococcus sp. oral taxon G63, Streptococcus sp. SHV515, Streptococcus suis, Streptococcus thermophilus, Streptococcus uberis, Streptococcus urinalis, Streptococcus vestibularis, Streptococcus viridans, Streptomyces albus, Streptomyces griseus, Streptomyces sp. 1 AIP_2009, Streptomyces sp. SD 511, Streptomyces sp. SD 524, Streptomyces sp. SD 528, Streptomyces sp. SD 534, Streptomyces thermoviolaceus, Subdoligranulum variabile, Succinatimonas hippei, Sutterella morbirenis, Sutterella parvirubra, Sutterella sanguinus, Sutterella sp. YIT 12072, Sutterella stercoricanis, Sutterella wadsworthensis, Synergistes genomosp. C1, Synergistes sp. RMA 14551, Synergistetes bacterium ADV897, Synergistetes bacterium LBVCM1157, Synergistetes bacterium oral taxon 362, Synergistetes bacterium oral taxon D48, Syntrophococcus sucromutans, Syntrophomonadaceae genomosp. P1, Tannerella forsythia, Tannerella sp. 6_1_58FAA_CT1, Tatlockia micdadei, Tatumella ptyseos, Tessaracoccus sp. oral taxon F04, Tetragenococcus halophilus, Tetragenococcus koreensis, Thermoanaerobacter pseudethanolicus, Thermobifida fusca, Thermofilum pendens, Thermus aquaticus, Tissierella praeacuta, Trabulsiella guamensis, Treponema denticola, Treponema genomosp. P1, Treponema genomosp. P4 oral clone MB2_G19, Treponema genomosp. P5 oral clone MB3_P23, Treponema genomosp. P6 oral clone MB4_G11, Treponema lecithinolyticum, Treponema pallidum, Treponema parvum, Treponema phagedenis, Treponema putidum, Treponema refringens, Treponema socranskii, Treponema sp. 6:H:D15A_4, Treponema sp. clone DDKL_4, Treponema sp. oral clone JU025, Treponema sp. oral clone JU031, Treponema sp. oral clone P2PB_53 P3, Treponema sp. oral taxon 228, Treponema sp. oral taxon 230, Treponema sp. oral taxon 231, Treponema sp. oral taxon 232, Treponema sp. oral taxon 235, Treponema sp. oral taxon 239, Treponema sp. oral taxon 247, Treponema sp. oral taxon 250, Treponema sp. oral taxon 251, Treponema sp. oral taxon 254, Treponema sp. oral taxon 265, Treponema sp. oral taxon 270, Treponema sp. oral taxon 271, Treponema sp. oral taxon 508, Treponema sp. oral taxon 518, Treponema sp. oral taxon G85, Treponema sp. ovine footrot, Treponema vincentii, Tropheryma whipplei, Trueperella pyogenes, Tsukamurella paurometabola, Tsukamurella tyrosinosolvens, Turicibacter sanguinis, Ureaplasma parvum, Ureaplasma urealyticum, Ureibacillus composti, Ureibacillus suwonensis, Ureibacillus terrenus, Ureibacillus thermophilus, Ureibacillus thermosphaericus, Vagococcus fluvialis, Veillonella atypica, Veillonella dispar, Veillonella genomosp. P1 oral clone MB5_P17, Veillonella montpellierensis, Veillonella parvula, Veillonella sp. 3_1_44, Veillonella sp. 6_1_27, Veillonella sp. ACP1, Veillonella sp. AS16, Veillonella sp. BS32b, Veillonella sp. ICM51a, Veillonella sp. MSA12, Veillonella sp. NVG 100cf, Veillonella sp. OK11, Veillonella sp. oral clone ASCA08, Veillonella sp. oral clone ASCB03, Veillonella sp. oral clone ASCG01, Veillonella sp. oral clone ASCG02, Veillonella sp. oral clone OH1A, Veillonella sp. oral taxon 158, Veillonellaceae bacterium oral taxon 131, Veillonellaceae bacterium oral taxon 155, Vibrio cholerae, Vibrio fluvialis, Vibrio furnissii, Vibrio mimicus, Vibrio parahaemolyticus, Vibrio sp. RC341, Vibrio vulnificus, Victivallaceae bacterium NML 080035, Victivallis vadensis, Virgibacillus proomii, Weissella beninensis, Weissella cibaria, Weissella confusa, Weissella hellenica, Weissella kandleri, Weissella koreensis, Weissella paramesenteroides, Weissella sp. KLDS 7.0701, Wolinella succinogenes, Xanthomonadaceae bacterium NML 03_0222, Xanthomonas campestris, Xanthomonas sp. kmd_489, Xenophilus aerolatus, Yersinia aldovae, Yersinia aleksiciae, Yersinia bercovieri, Yersinia enterocolitica, Yersinia frederiksenii, Yersinia intermedia, Yersinia kristensenii, Yersinia mollaretii, Yersinia pestis, Yersinia pseudotuberculosis, Yersinia rohdei, Yokenella regensburgei, Zimmermannella bifida, Zymomonas mobilis, Blautia massiliensis, Paraclostridium benzoelyticum, Dielma fastidiosa, Longicatena caecimuris, and Veillonella tobetsuensis.

In some embodiments, the bacterium is selected from the group consisting of Bacillus, Bacteroides, Bifidobacterium, Clostridium, Corynebacterium, Escherichia, Lactobacillus, Lactococcus, Pseudomonas, Streptomyces, or Mycobacterium. In some embodiments, the bacterium is Escherichia coli strain Nissle, MG1655, or S1030.

### EXAMPLES

### Example 1: Methods

Materials: Low density polyethylene LDPE (ET31-FM-000151, 50 μm thick), Polytetrafluoroethylene PTFE (FP30-FM-000250, 50 μm thick), Poly-L-lactic acid PLLA (ME33-FM-000150, 50 μm thick), Polyethylene terephthalate PET (ES30-FM-000150, 50 μm thick), Cellulose acetate CA (AC31-FM-000151, 50 μm thick) and Polypropylene PP (PP30-FM-000250, 50 μm thick) were obtained from Goodfellow Corporation. Polyvinyl chloride PVC (S-16280, 15 μm thick) was obtained from Uline. Polyvinyl alcohol-Superpunch PVA-Sp (ASIN: BO1M11T6U5; a water soluble stabilizer for embroidery and it is claimed to be made from 100% PVA and thus it dissolves completely in water), Polyvinyl alcohol-McKesson PVA-Mc (ASIN: B01ETFMUH2; a hot water soluble bag, which is also claimed to be made from PVA, but it does not dissolve completely in water at room temperature, probably because PVA might be blended with other components.) and Silicone mats (ASIN: B09SPB72TT) were obtained from Amazon. Aluminum foil, Parafilm, Toilet paper and Kimwipes were obtained from Reynolds Consumer Products, Bemis Company Inc., Signature Select and Kimberly-Clark Corporation, respectively. Glycerol (G9012) and Sodium dodecyl sulfate SDS (S0295) were obtained from Sigma-Aldrich and Teknova, respectively.

Plasmids to produce MECHS: pET21d plasmid was cloned with the Curli operon genes csgA, csgB, csgC, csgE, csgF and csgG that encodes the proteins necessary for biosynthesis of Curli nanofibers and it is labelled as pET21d-CsgA. The genes encoding the SpyTag peptide and SpyCatcher protein derived from Keeble, A. H. et al. (Proc. Natl. Acad. of Sci. 116, 26523-26533-2019) were fused to the C-terminus of CsgA with an intervening 36 amino acid flexible linker to obtain the plasmids pET21d-CsgA-SpyTag and pET21d-CsgA-SpyCatcher, respectively (Table 2). The gene encoding the Spacer, an intrinsically disordered protein (Pramanik, U., et al. Chem. Phys. Lett. 762, 138105-2021), was inserted between the linker and the SpyTag or the SpyCatcher to obtain pET21d-CsgA-Spacer-SpyTag and pET21d-CsgA-Spacer-SpyCatcher, respectively. The genes were synthesized (Integrated DNA Technologies) and cloned into pET21d vector using isothermal Gibson assembly (New England Biolabs).

TABLE 1
Amino Acid sequences of
peptide/protein domains of MECHS variants.

| Protein/ | |
|---|---|
| Peptide | Amino Acid Sequence |
| CsgA | GVVPQYGGGGNHGGGGNNSGPNSELNIYQYGG |
| | GNSALALQTDARNSDLTITQHGGGNGADVGQG |
| | SDDSSIDLTQRGFGNSATLDQWNGKNSEMTVK |
| | QFGGGNGAAVDQTASNSSVNVTQVGFGNNATA |
| | HQY (SEQ ID NO: 1) |
| Linker | GGSGSSGSGGSGGGSGSSGSGGSGGGSGSSGS |
| | GGSG (SEQ ID NO: 2) |
| SpyTag | RGVPHIVMVDAYKRYK (SEQ ID NO: 3) |
| SpyCatcher | VTTLSGLSGEQGPSGDMTTEEDSATHIKFSKR |
| | DEDGRELAGATMELRDSSGKTISTWISDGHVK |
| | DFYLYPGKYTFVETAAPDGYEVATPIEFTVNE |
| | DGQVTVDGEATEGDAHT (SEQ ID NO: 4) |
| Spacer | KVLILACLVALALARETIESLSSSEESITEYK |
| | QKVEKVKHEDQQQGEDEHQDKIYPSFQPQPLI |
| | YPFVEPIPYGFLPQNILPLAQPAVVLPVPQPE |
| | IMEVPKAKDTVYTKGRVMPVLKSPTIPFFDPQ |
| | IPKLTDLENLHLPLPLLQPLMQQVPQPIPQTL |
| | ALPPQPLWSVPQPKVLPIPQQVVPYPQRAVPV |
| | QALLLNQELLLNPTHQIYPVTQPLAPVHNPIS |
| | V (SEQ ID NO: 5) |

TABLE 2
Genes and their DNA sequence.

| Gene | DNA Sequence |
|---|---|
| CsgA | GGTGTTGTTCCTCAGTACGGCGGCGGCGGTAACCACGGTG |
| | GTGGCGGTAATAATAGCGGCCCAAATTCTGAGCTGAACAT |
| | TTACCAGTACGGTGGCGGTAACTCTGCACTTGCTCTGCAA |
| | ACTGATGCCCGTAACTCTGACTTGACTATTACCCAGCATG |
| | GCGGCGGTAATGGTGCAGATGTTGGTCAGGGCTCAGATGA |
| | CAGCTCAATCGATCTGACCCAACGTGGCTTCGGTAACAGC |
| | GCTACTCTTGATCAGTGGAACGGCAAAAATTCTGAAATGA |
| | CGGTTAAACAGTTCGGTGGTGGCAACGGTGCTGCAGTTGA |
| | CCAGACTGCATCTAACTCCTCCGTCAACGTGACTCAGGTT |
| | GGCTTTGGTAACAACGCGACCGCTCATCAGTAC (SEQ |
| | ID NO: 6) |
| Linker | GGTGGATCTGGTAGCAGCGGCTCTGGTGGTTCTGGGGGCG |
| | GAAGTGGCTCCTCTGGGAGCGGGGGGTCGGGTGGTGGCTC |
| | GGGTTCATCTGGTAGTGGCGGTTCGGGT (SEQ ID |
| | NO: 7) |
| SpyTag | CGCGGCGTGCCGCATATTGTGATGGTGGATGCGTATAAAC |
| | GCTATAAA (SEQ ID NO: 8) |
| SpyCatcher | GTGACCACCCTGAGCGGCCTGAGCGGCGAACAGGGCCCGA |
| | GCGGCGATATGACCACCGAAGAAGATAGCGCGACCCATAT |
| | TAAATTTAGCAAACGCGATGAAGATGGCCGCGAACTGGCG |
| | GGCGCGACCATGGAACTGCGCGATAGCAGCGGCAAAACCA |
| | TTAGCACCTGGATTAGCGATGGCCATGTGAAAGATTTTTA |
| | TCTGTATCCGGGCAAATATACCTTTGTGGAAACCGCGGCG |
| | CCGGATGGCTATGAAGTGGCGACCCCGATTGAATTTACCG |
| | TGAACGAAGATGGCCAGGTGACCGTGGATGGCGAAGCGAC |
| | CGAAGGCGATGCGCATACC (SEQ ID NO: 9) |
| Spacer | ATGAAAGTGCTGATTCTGGCGTGCCTGGTGGCGCTGGCGC |
| | TGGCGCGCGAAACCATTGAAAGCCTGAGCAGCAGCGAAGA |
| | AAGCATTACCGAATATAAACAGAAAGTGGAAAAAGTGAAA |
| | CATGAAGATCAGCAGCAGGGCGAAGATGAACATCAGGATA |
| | AAATTTATCCGAGCTTTCAGCCGCAGCCGCTGATTTATCC |
| | GTTTGTGGAACCGATTCCGTATGGCTTTCTGCCGCAGAAC |
| | ATTCTGCCGCTGGCGCAGCCGGCGGTGGTGCTGCCGGTGC |
| | CGCAGCCGGAAATTATGGAAGTGCCGAAAGCGAAAGATAC |
| | CGTGTATACCAAAGGCCGCGTGATGCCGGTGCTGAAAAGC |
| | CCGACCATTCCGTTTTTTGATCCGCAGATTCCGAAACTGA |
| | CCGATCTGGAAAACCTGCATCTGCCGCTGCCGCTGCTGCA |
| | GCCGCTGATGCAGCAGGTGCCGCAGCCGATTCCGCAGACC |
| | CTGGCGCTGCCGCCGCAGCCGCTGTGGAGCGTGCCGCAGC |
| | CGAAAGTGCTGCCGATTCCGCAGCAGGTGGTGCCGTATCC |
| | GCAGCGCGCGGTGCCGGTGCAGGCGCTGCTGCTGAACCAG |
| | GAACTGCTGCTGAACCCGACCCATCAGATTTATCCGGTGA |
| | CCCAGCCGCTGGCGCCGGTGCATAACCCGATTAGCGTG |
| | (SEQ ID NO: 10) |

Cell strain to produce MECHS: The plasmids pET21d-CsgA, pET21d-CsgA-SpyTag, pET21d-CsgA-SpyCatcher, pET21d-CsgA-Spacer-SpyTag and pET21d-CsgA-Spacer-SpyCatcher were separately transformed into PQN4, an E. coli cell strain derived from LSR10 (MC4100, ΔcsgA, λ(DE3), Cam^{R}) with the deletion of Curli operon (ΔcsgBACEFG) to produce the corresponding MECHS.

Cell culture to produce MECHS (CsgA): pET21d-CsgA plasmid was transformed into PQN4 and streaked onto lysogeny broth (LB) agar plate containing 100 μg mL⁻¹ carbenicillin and 0.5% glucose (m v⁻¹) for catabolite repression of T7RNAP and incubated overnight at 37° C. A single colony of PQN4-pET21d-CsgA was picked from the agar plate and cultured at 37° C. in 5 mL LB media, 100 μg mL⁻¹ carbenicillin and 2% glucose (m v⁻¹). The overnight culture was transferred to a fresh 500 mL LB media containing 100 μg mL⁻¹ carbenicillin and cultured for 48 h in incubator shakers (225 rpm, 37° C.) to express the CsgA Curli protein nanofibers.

Cell culture to produce the covalently crosslinked MECHS (CL1 and CL2): For Covalently Crosslinked-1 (CL1), the plasmids pET21d-CsgA-SpyTag and pET21d-CsgA-SpyCatcher were separately transformed into PQN4 and streaked onto lysogeny broth (LB) agar plates containing 100 μg mL⁻¹ carbenicillin and 0.5% glucose (m v⁻¹) for catabolite repression of T7RNAP and incubated overnight at 37° C. A single colony was picked from the agar plates of PQN4-pET21d-CsgA-SpyTag and PQN4-pET21d-CsgA-SpyCatcher and cultured separately at 37° C. in 5 mL LB media, 100 μg mL⁻¹ carbenicillin and 2% glucose (m v⁻¹). The overnight cultures of PQN4-pET21d-CsgA-SpyTag and PQN4-pET21d-CsgA-SpyCatcher were transferred to a fresh 500 mL LB media containing 100 μg mL⁻¹ carbenicillin and co-cultured for 48 h in incubator shakers (225 rpm, 37° C.) to express and covalently crosslink the engineered Curli protein nanofibers. Similarly, the plasmids pET21d-CsgA-Spacer-SpyTag and pET21d-CsgA-Spacer-SpyCatcher were utilized for Covalently Crosslinked-2 (CL2) MECHS.

Biofabrication of MECHS: The 48 h cell culture (500 mL) of PQN4-pET21d-CsgA (CsgA) was centrifuged (5000 g, 10 min) to pelletize the Curli biomass, which was then washed with 250 mL of deionized water by centrifuging (5000 g, 10 min) to remove the residual quantities of culture media. 1 g (wet pellet) of Curli biofilm biomass was first dispersed in 5 mL of deionized water and subsequently added with 5 mL of 1%, 2%, 3%, 4% or 5% (w v⁻¹) of sodium dodecyl sulfate (SDS, serves as a gelator and also helps to obtain the transparent MECHS films by taking away the brown-yellowish color of cell pellet into the supernatant), which was then mixed on a shaker for 2 h at room temperature. The resulting gelatinous biomass was washed with 10 mL of deionized water twice by centrifuging (5000 g, 10 min) to remove the soluble biomolecules and the excess SDS. This SDS treated gelatinous biomass was casted and ambient dried on a silicone mold to obtain the MECHS films that were brittle.

To realize the flexible films of MECHS, the 3% SDS treated gelatinous biomass of PQN4-pET21d-CsgA (CsgA) was added with 5 mL of 1%, 2%, 3%, 4%, or 5% (w v⁻¹) of glycerol (serves as a plasticizer) and mixed on a shaker for 1 h at room temperature. The glycerol treated and centrifuged (5000 g, 10 min) biomass was casted on a silicone mold and ambient dried to obtain the flexible MECHS films.

Similarly, to realize the Covalently Crosslinked (CL1 and CL2) films of MECHS, 5 mL of 3% SDS and 5 mL of 3% glycerol treated Curli biomass was utilized. For all constructs, a minimum of ten replicates were tested.

Field-emission scanning electron microscopy (FESEM) sample preparation and imaging: 100 μL of cell culture was vacuum filtered on a membrane (0.22 μm pore size, Millipore GTTP02500) and washed with 100 L of deionized water thrice. The samples were fixed by immersing in 2 mL 1:1 mixture of 4% (w v⁻¹) glutaraldehyde and 4% (w v⁻¹) paraformaldehyde at room temperature, overnight. The samples were gently washed with water, and the solvent was gradually exchanged to ethanol (200 proof) with an increasing ethanol 15-minute incubation step gradient [25, 50, 75 and 100% (v v⁻¹) ethanol]. The samples were then dried in a critical point dryer, placed onto SEM sample holders using silver adhesive (Electron Microscopy Sciences) and sputtered until they were coated in a 10-20 nm layer of Pt/Pd. Whereas the films of MECHS were directly sputter coated with 10-20 nm layer of Pt/Pd without critical point drying. Images were acquired using a Zeiss Gemini 360 FESEM equipped with a field emission gun operating at 5-10 kV. Representative images from three independent samples were reported.

Energy dispersive x-ray xnalysis (EDAX): Oxford Instruments Ultim Max EDS equipped with AZtecLive software attached to Zeiss Gemini 360 FESEM was utilized to detect the elements as well as determine their composition using factory standards. EDS spectra were recorded on sample's surface with the lateral dimensions of 225 μm by 170 μm. Data from three independent samples were reported.

Optical images: Optical images were acquired using a Canon EOS Rebel SL3 Digital SLR Camera equipped with XIT 58 mm 0.43 Wide Angle Lens and XIT 58 mm 2.2× Telephoto Lens. Representative images from three independent samples were reported.

Tensile tests: Tensile measurements of MECHS, commercially available plastics, bioplastics, and all other materials mentioned in this report were performed using a DHR-3 rheometer (TA Instruments) under ambient laboratory conditions. Films with the lateral dimensions of 4 cm by 0.5 cm under a constant linear deformation of 1 μm s⁻¹ were utilized for tensile tests. A minimum of five samples were tested for each type.

Film thickness: The thickness of the films was measured using a contact profilometer, Dektak 3ST equipped with a 2.5 μm stylus having a vertical resolution of 1A. A minimum of three tests were performed for each sample.

Large prototypes: The MECHS prototype of 50 cm×5 cm lateral dimension was fabricated from 6 L cultures of PQN4-pET21d-CsgA (obtained by using 3% SDS and 3% glycerol treatment), whereas the 15 cm×10 cm and the detergent pod prototypes were obtained from that of 4 and 3 L cultures, respectively.

Healing: The films of MECHS (PQN4-pET21d-CsgA, obtained by using 3% SDS and 3% glycerol) were cut using scissors and ˜10 μL of deionized water was added at the cut site and subsequently dried at ambient laboratory conditions to heal the cut. A minimum of three samples were tested. Similarly, MECHS films of 0.5 cm by 5 cm were welded by using ˜10 μL of deionized water and subsequently dried at ambient laboratory conditions.

Biodegradation: A commercially available odorless organic humus compost named Fishnure (Amazon, ASIN: B086KXT5TQ), which is made from fish manure was utilized for the biodegradation test. Samples with the lateral dimensions of 5 cm×5 cm were buried in a tray containing 3.5 kg of Fishnure. The biodegradation experiment was conducted in a mini greenhouse (Amazon, ASIN: B01D7GHEES) setup (exposed to direct/indirect sunlight through the large windows of the laboratory), wherein a temperature of 20° C. and a relative humidity of 80% was maintained. The films of MECHS degraded completely in 15 days in a freshly opened bag of Fishnure. In another biodegradation experiment, a dry (by placing in the mini greenhouse setup for 50 days) Fishnure was utilized and under these conditions, films of MECHS degraded completely in 75 days. A minimum of three samples were tested for each type.

Congo Red assay: 1 mL of cell culture (as described above: 48 h, 500 mL at 37° C.) was pelleted by centrifuging (6000 g, 10 min) and the resulting cell pellet was incubated with 1 mL of 0.004% (w v⁻¹) Congo Red dye for 10 min. The dye treated cell culture was pelletized by centrifuging (6000 g, 10 min) and the resulting supernatant (200 μL) was utilized to measure the absorbance at 480 nm in a plate reader. The net Congo Red absorbance of Curli in CsgA, CL1 and CL2 were determined by subtracting the absorbance values of cell pellet having a sham plasmid (without Curli operon), to account for the non-specific binding to other biomolecules.

To estimate the Curli nanofibers produced, we utilized 0.004% (w v⁻¹) Congo Red dye to prepare a standard curve for various concentrations of purified CsgA. Herein, C-terminal His-tagged CsgA (CsgA-His) was expressed and purified using Ni-NTA (Nickel-nitrilotriacetic acid resin) column. However, after eluting the CsgA-His with the elution buffer, the buffer was exchanged with water using 10 kDa Amicon centrifugal filter. This buffer exchange facilitates fibrillation of CsgA-His and the resulting pellet (wet weight) was utilized for the CsgA (CsgA-His) Congo Red standard curve. A minimum of three samples were tested for each type.

Statistics and reproducibility: All experiments presented in this article were repeated at least three times (n≥3) on distinct samples or biological replicates, as clearly specified in the figure legends or the relevant Methods sections. In all cases, data are presented as the mean and standard deviation. GraphPad PRISM 8, OriginPro 2024, Oxford Instruments Ultim Max EDS AZtecLive software, TRIOS software V5.2, Adobe Photoshop 2024 and Adobe Illustrator 2024 were utilized for plotting and analyzing data. For micrographs and optical images, we present representative images.

### Example 2: Biofabrication of MECHS

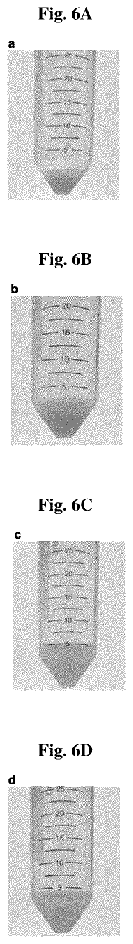

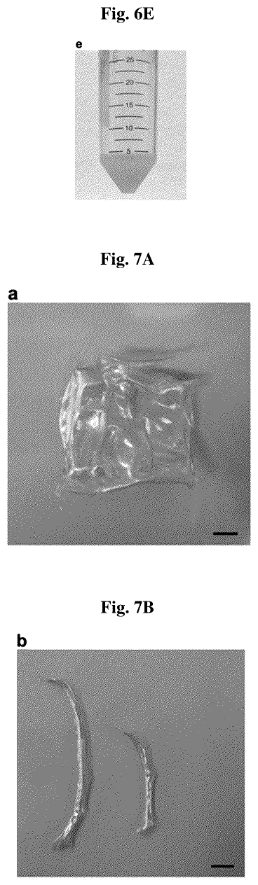

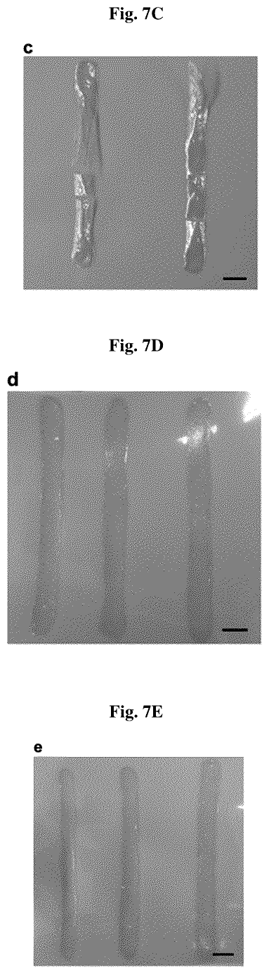

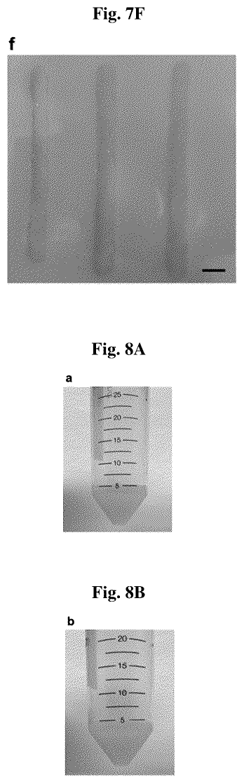

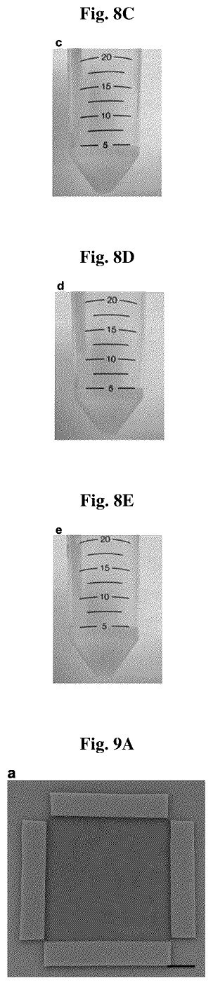

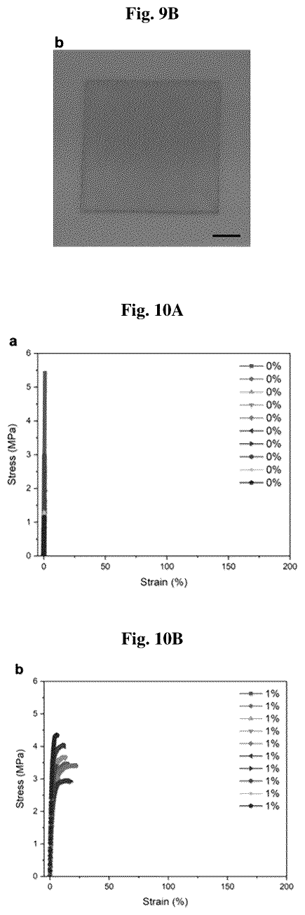

MECHS is fabricated from a combination of whole E. coli cells and engineered recombinant Curli nanofibers. Curli are an extracellular matrix component of microbial biofilms and are composed of nanofibers self-assembled from a protein building block, CsgA (FIGS. 1A-1D, Tables 1 and 2). Curli nanofibers are resistant to heat, solvents, pH, detergents, and denaturants, and thus serve as a good biopolymeric scaffold for robust materials. To express the recombinant Curli nanofibers, we used an E. coli strain that we previously developed (PQN4), in which the chromosomal Curli genes (csgBAC, csgDEFG) have been deleted. PQN4 was transformed with a pET21d plasmid vector encoding a synthetic Curli operon, csgBACEFG, containing all the genes necessary for CsgA production, secretion, and extracellular assembly. In a typical biofabrication of MECHS, the Curli-containing E. coli biomass was treated with 1-5% (w v⁻¹) of sodium dodecyl sulfate (SDS) to obtain a gelatinous substance, which enables facile casting in a silicone mold. Ambient drying in the mold resulted in films that were brittle and, in some cases, (1% and 2% SDS) convoluted (FIG. 6 and FIG. 7). To achieve flexible MECHS films, we added glycerol (1-5% w v⁻¹), a plasticizer commonly used with bioplastics, to the gelatinous Curli biomass prior to casting (FIG. 8 and FIG. 9).

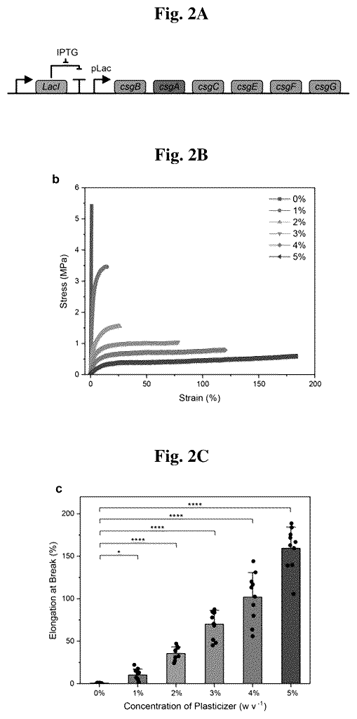

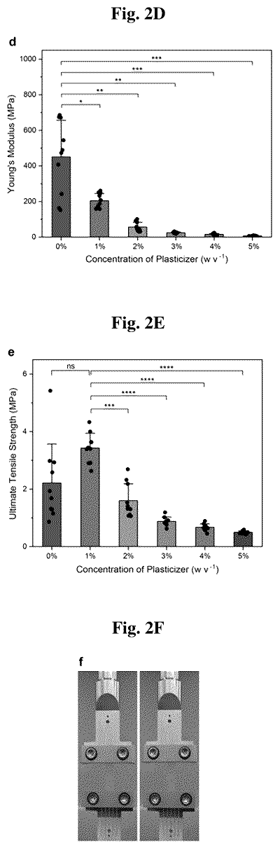

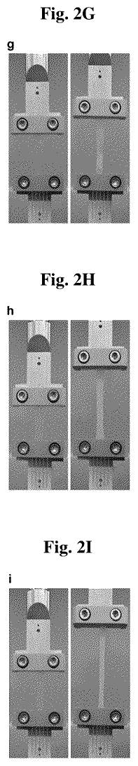

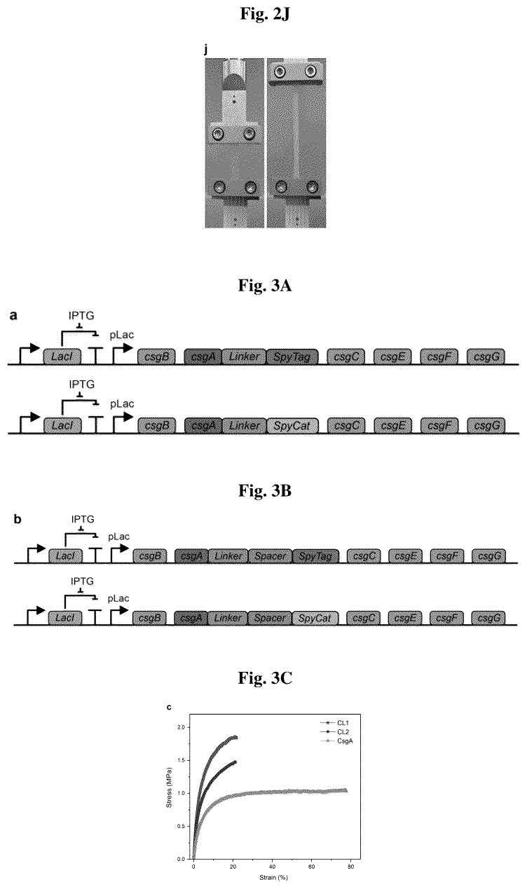

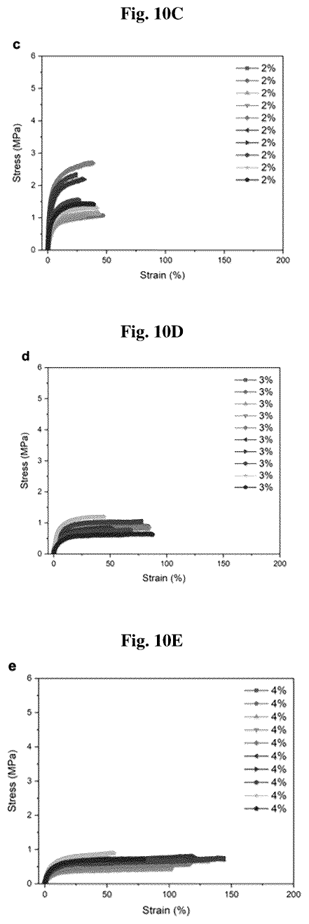

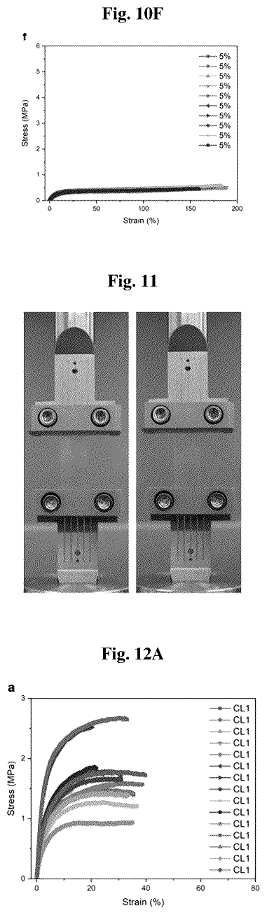

MECHS films that had been pre-treated only with SDS (i.e., gelator) and no glycerol (i.e., plasticizer), were brittle as measured by tensile mechanical tests, with elongation at break values of 0.6±0.4% (FIGS. 2A-2E, FIG. 10A, and FIG. 11). With 1% plasticizer, the elongation at break was found to increase considerably to 10.2±6.9% (FIGS. 2B-2F and FIG. 10B). Similarly, as the plasticizer content increased to 2%, 3%, 4% and 5%, the elongation at break increased significantly to 35.5±7.7%, 70.1±16.3%, 101.9±28.8%, and 159.3±25% respectively (FIGS. 2B-2E, 2G-2J and FIGS. 10C-10F). On the other hand, the corresponding Young's modulus decreased from 450±206.4 MPa to 6.6±1.7 MPa as the plasticizer amount increased (FIG. 2D). Ultimate tensile strength values of MECHS films also decreased with increasing plasticizer (FIG. 2E). Overall, our method further streamlines the fabrication of flexible MECHS films from our previous demonstrations by casting directly from whole microbial biomass, without the need for filtration and extensive washing. However, it also provides an opportunity to tailor their mechanical properties by two orders of magnitude by inclusion of the engineered Curli nanofibers and a plasticizer.

### Example 3: Genetically Engineered Curli Nanofibers to Tailor the Mechanical Properties

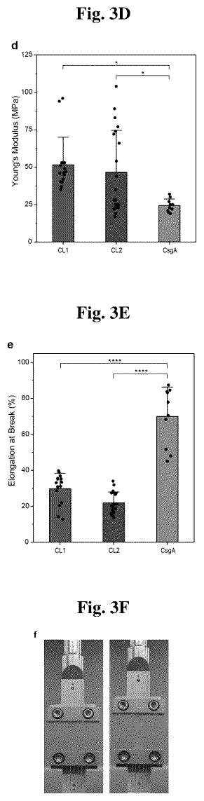

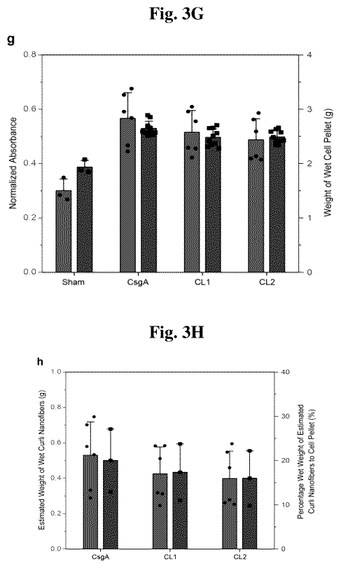

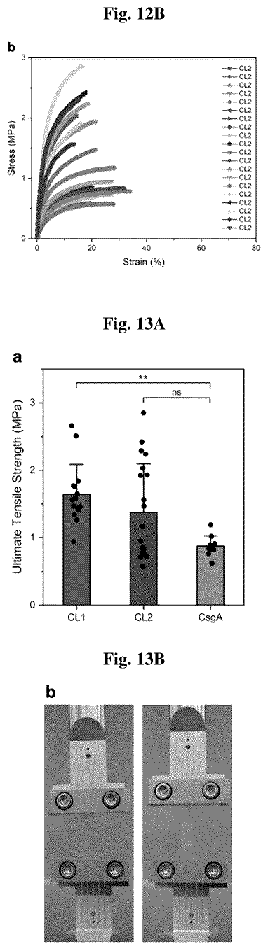

We genetically engineered the Curli nanofibers to further modulate the mechanical properties of MECHS. We developed Biofilm Integrated Nanofiber Display (BIND), wherein genetic fusions to CsgA are used to modulate material properties of assembled Curli nanofibers. During extracellular self-assembly, the robust, β-helical blocks of CsgA fusions, stack on top of each other to form functional Curli nanofibers with the desired peptide/protein fusions displayed on their surface. We used the genetic programmability of BIND to increase the stiffness of MECHS through covalent crosslinking. To achieve this, we utilized the third generation of split proteins derived from the adhesion domain, CnaB2 of Streptococcus pyogenes (SpyTag/SpyCatcher), wherein a spontaneous reaction between the side chains of lysine and aspartic acid residues results in the formation of an isopeptide bond. This amide bond formation was reported to have a high reactivity with >90% completion in 15 min at 10 nM concentration, and that for 10 μM, the half-time was less than 30 s. Moreover, the reaction does not require any activating groups and is highly specific even in various complex biological media. SpyTag and SpyCatcher were each genetically grafted to CsgA via a linker to obtain CsgA-SpyTag and CsgA-SpyCatcher (FIG. 3A). These two CsgA constructs were expressed from separate plasmids in a co-culture and the resulting Curli biomass was used to fabricate MECHS films (denoted as CL1, FIG. 1B). The tensile tests of CL1 showed that their Young's modulus (51.6±18.4 MPa) and ultimate tensile strengths (1.6±0.4 MPa) were twice that of CsgA only (i.e., not crosslinked) based MECHS films, (FIGS. 3C, 3D, 3F, FIGS. 12A and 13A). However, the elongation at break of CL1 was found to reduce to 29.8±8.6% (FIG. 3E). We also tried analogous experiments with a large spacer (disordered protein domain of 225 amino acids) in between CsgA and the SpyTag/SpyCatcher domains (FIG. 3B and FIG. 1C). We introduced the large spacer for two reasons. 1) To verify if the observed increase in stiffness of CL1 was due to the covalent crosslinking of SpyTag and SpyCatcher. 2) To test if an intrinsically disordered large protein can modulate the mechanical properties such as stiffness, toughness and elongation at break. MECHS films with this composition (i.e., CL2) were also found to have a Young's modulus (46.6±27.9 MPa), ultimate tensile strength (1.4±0.7 MPa) and elongation at break (21.9±6%), in the same range as that of CL1 (FIGS. 3C-3E, FIGS. 12B and 13A, and FIG. 13B). The inter-fibrillar interactions of Curli nanofibers in CsgA are that of relatively weaker supramolecular interactions, whereas for CL1 and CL2, the inter-fibrillar covalent crosslinking of Curli nanofibers is expected to resist the deformation of MECHS films leading to increased Young's modulus and ultimate tensile strength. However, this was achieved at the expense of elongation at break for CL1 and CL2 films. Although the covalent crosslinks enhance the stiffness of CL1 and CL2, we speculate that the softer biomass in the interstices between Curli aggregates provide alternate pathways for crack propagation. Moreover, the slight decrease in the Young's modulus and the ultimate tensile strength of CL2 in comparison to CL1 might be attributed to the effect of the disordered spacer domain. We reason that an even bigger spacer domain might lead to significant reductions to stiffness and enhanced extensibility.

### Example 4: Composition and Morphological Analysis

Given the highly heterogeneous nature of the whole biomass that forms MECHS, we wanted to perform a detailed compositional analysis to understand the effects of various components therein. We focused on determining the amounts of Curli biomass, gelator, and plasticizer in the final product, which may not be obvious from the fabrication protocol of MECHS. For example, treatment of the wet biomass with 1-5% gelator and/or plasticizer does not mean that the final MECHS film contains 1-5% gelator and/or plasticizer by mass, since only a portion of the original SDS and glycerol will associate with the cell pellet and the rest will be discarded with the supernatant, prior to film casting.

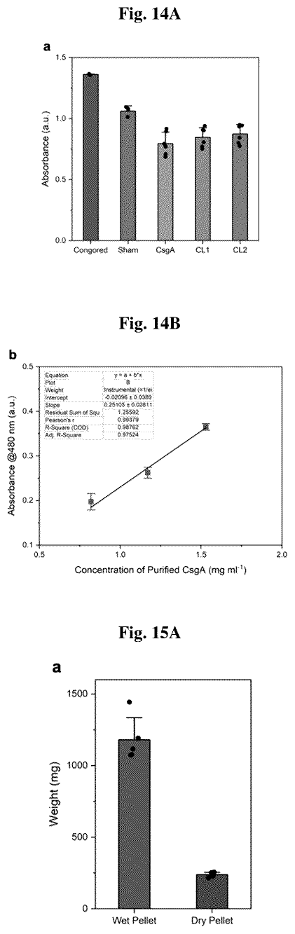

We first focused on estimating the amount of Curli nanofibers present in the films on a per-weight basis using a standard Congo Red pull-down assay for Curli quantification (FIG. 3G and FIG. 14A). These relative amounts of Curli were converted to absolute mass estimates with a calibration curve generated using purified Curli nanofibers (wet weights of CsgA fused with His-tag i.e., CsgA-His). We estimated that 500 mL cultures of CsgA, CL1, and CL2 produced 530±188 mg, 431±159 mg, and 399±154 mg of Curli nanofibers, respectively (FIG. 3H and FIG. 14B). The wet weights of whole cell pellets obtained from 500 mL cultures of CsgA, CL1 and CL2 were found to be 2647±130 mg, 2483±157 mg, 2490±118 mg, respectively (FIG. 3G). Thus, we could estimate the percent of wet weight contributed by Curli nanofibers for each construct (FIG. 3H). Notably, it is possible that the fused SpyTag/SpyCatcher domains may interfere with Congo Red binding, leading to an underestimation of Curli nanofiber yields. On the other hand, 500 mL cultures of PQN4 with a sham plasmid (no Curli operon) were found to have a wet cell pellet weight of 1936±123 mg (FIG. 3G). It is interesting to note that the differences in wet pellet mass between Curli-producing and sham plasmids roughly corresponds to the mass of Curli nanofibers in each culture, calculated from the calibrated Congo Red binding assay (FIGS. 3G, 3H).

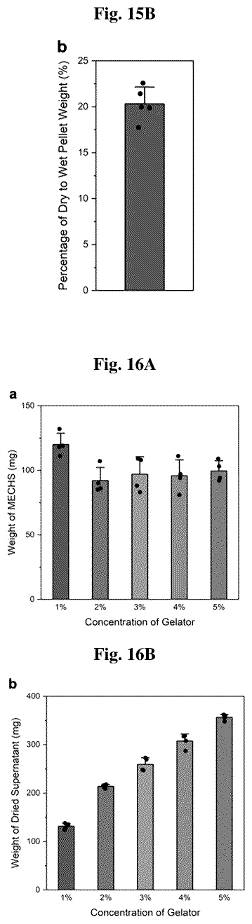

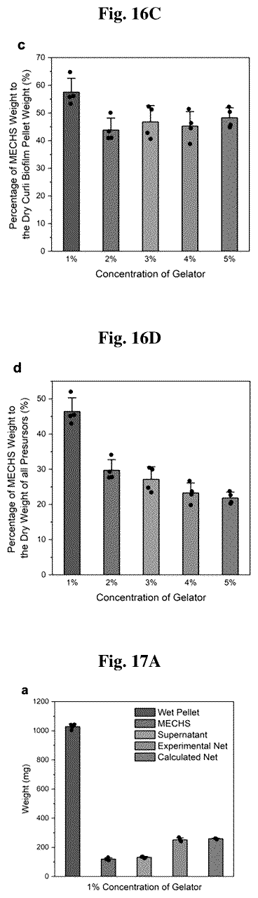

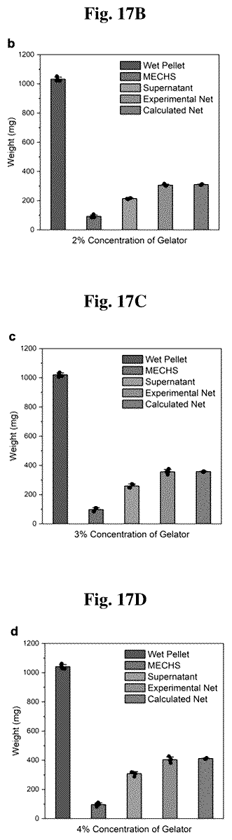

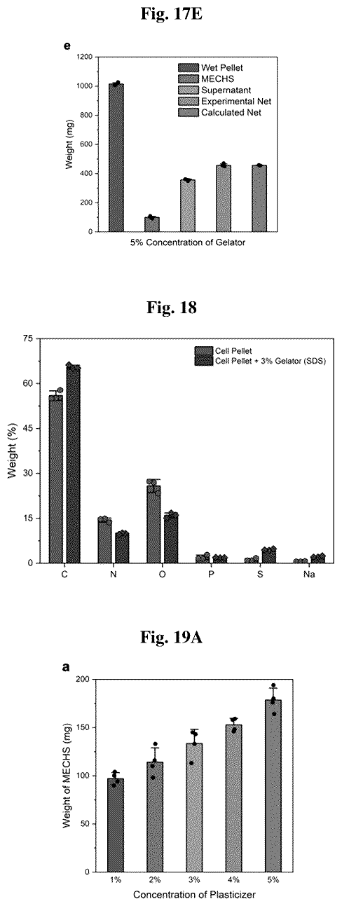

We then set out for an extensive weight analysis to better understand the composition and the effect of various steps involved in the fabrication of MECHS. First, we determined that the ambient drying of the wet pellet of Curli biofilm (without the treatment of gelator and plasticizer) results in a dry pellet with a weight percentage (dry to wet pellet) of 20.3±1.8% (FIGS. 15A, 15B). The dry weight of MECHS films obtained after treatment of 1-5% of gelator was found to be about 100 mg, while the dry weight of the supernatant (collected from all the SDS treatment and water washings of cell pellet) was found to increase linearly (FIGS. 16A-16D). It is to be noted that the experimentally obtained sum of weights of MECHS and the corresponding dry supernatant were consistent with their theoretically calculated weights (FIGS. 17A-17E). Further, we estimated that the weights of the gelator-treated MECHS films were nearly half of the estimated dry weight (20.3% of wet pellet weight) of Curli biomass (FIG. 16C). Similarly, the weights of MECHS films obtained from 1% and 2% gelator were nearly 45% and 30%, respectively, of the estimated total weight of all precursors, whereas that for 3-5% gelator were about 25% (FIG. 16D). These results also suggests that the convoluted MECHS films obtained from 1% and 2% gelator upon drying could be attributed to incorporation of more cellular biomass into the films, while the 3-5% gelator might extract more cellular components like lipids into the supernatant (FIGS. 27B, 7C). Moreover, it is to be noted that unlike 1% and 2% of gelator concentrations, the 3-5% of gelator leads to better gelatinous Curli biomass (FIG. 6). As the percentage weight of MECHS with respect to the dry weight of Curli biofilm remains at around 45%, it suggests that the higher gelator (3-5%) content might not lead to additional loss of biomass into supernatant (FIG. 16C). This latter inference is also supported by the fact that weight of dried supernatant increases in steps of ˜50 mg, which is consistent with the expected increase in the theoretical weights of gelator (e.g., 5 mL of 1% accounts for 50 mg) (FIG. 16B).

As noted above, 3-5% gelator-treated MECHS comprises of nearly 45% dry weight of the whole cell pellet, then we reasoned that by determining the amount of SDS, we could estimate the total (cellular and Curli) biomass in the MECHS (FIG. 16C). By using Energy Dispersive X-ray Analysis (EDAX) we found out that for 3% gelator-treated MECHS, the weight percentage of Sodium and Sulfur elements were 2.2±0.2% and 4.5±0.3%, respectively, whereas the same elements for the Curli biofilm cell pellet (without SDS treatment) were 0.6±0.1% and 1.2±0.5%, respectively (FIG. 18). Using this data, we estimate that for 3% gelator-treated films, roughly 5% (˜1.6% Sodium and ˜3.3% Sulfur) of MECHS weights could comprise of SDS (FIG. 16A, 16C). Therefore, we can estimate that about 40% of the total cellular and Curli biomass might get utilized to form the gelator-treated MECHS.

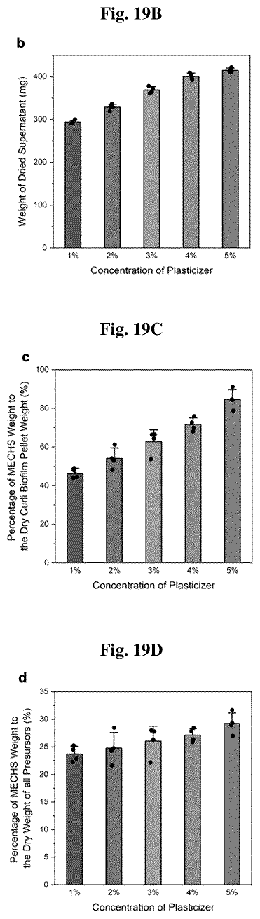

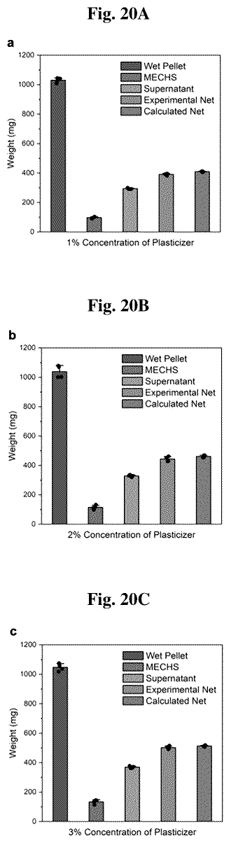

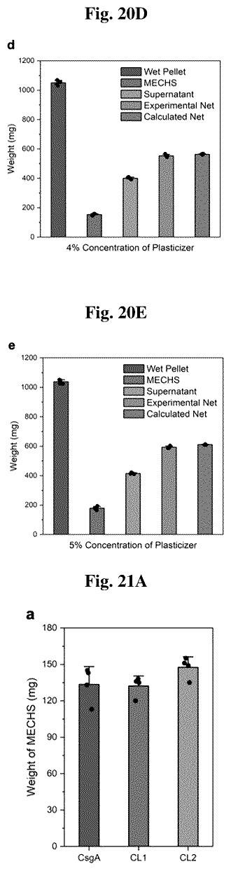

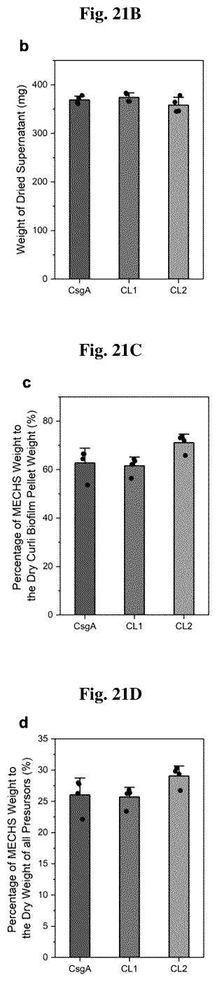

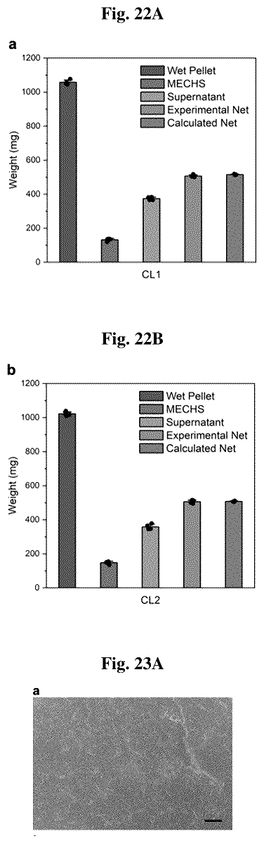

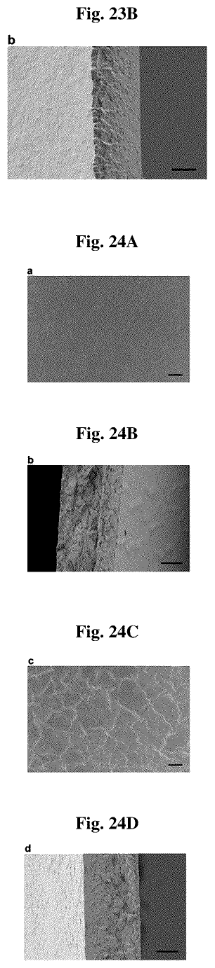

On the other hand, based on the weights of plasticizer-treated MECHS films and their corresponding dry supernatant weights, we could estimate that 15-20% of the total plasticizer utilized might get incorporated into MECHS, assuming that no additional biomass was lost to the supernatant during this phase of fabrication (FIGS. 16, 19 and 20). In addition, the weights of MECHS films of CsgA, CL1 and CL2 and their dried supernatants were in the same range, which further validates that the covalent crosslinking in CL1 and CL2, leads to increased stiffness and not due to any variations in the plasticizer amounts (FIGS. 21 and 22).

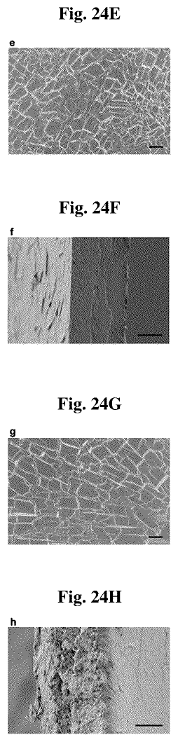

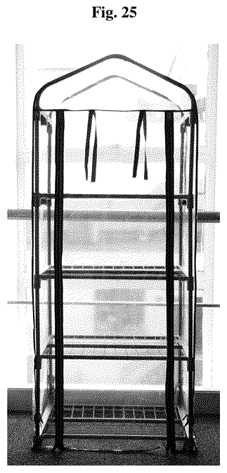

Field Emission Scanning Electron Microscopy (FESEM) images from cultures of CL1, and CL2 showed aggregated mats of material, presumably due to nanofiber bundling promoted by the SpyTag/SpyCatcher covalent crosslinking. Images obtained from CsgA cultures did not show such aggregation (FIG. 3I). FESEM images of MECHS (top and side view) further showed that the Curli biomass is densely packed to form continuous films (FIG. 3I, FIGS. 23 and 24).

### Example 5: Compostability, Scalability and Mechanical Landscape

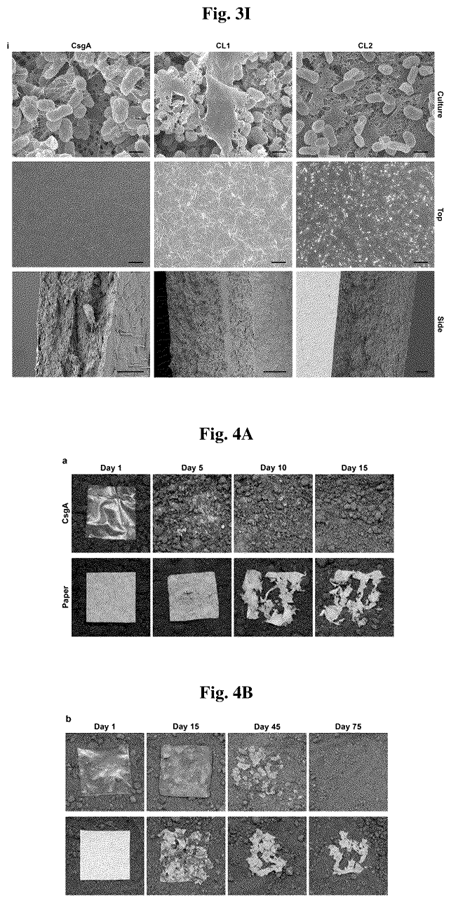

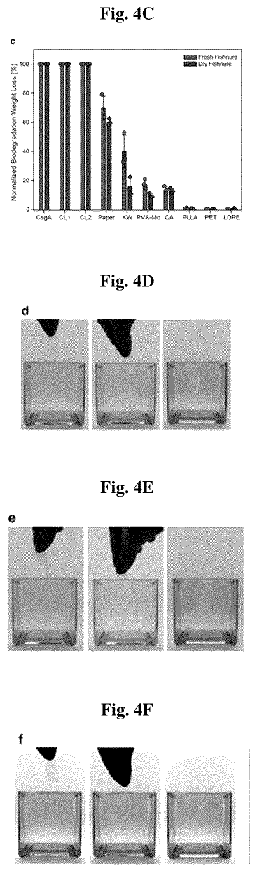

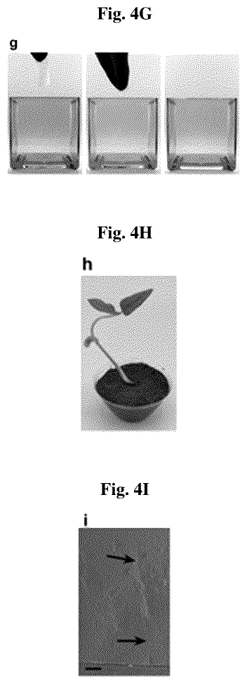

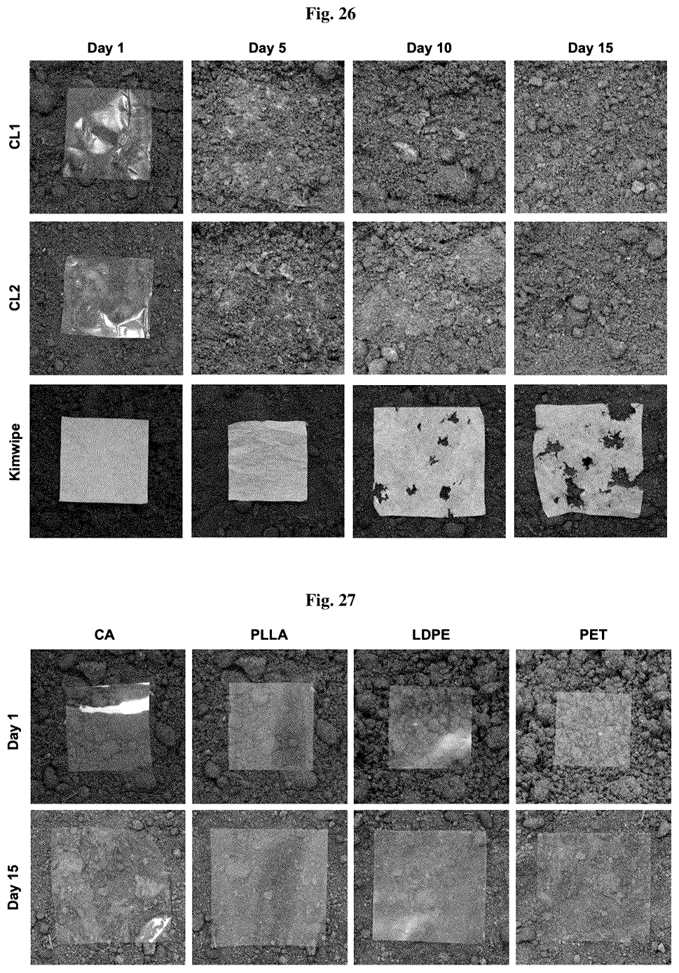

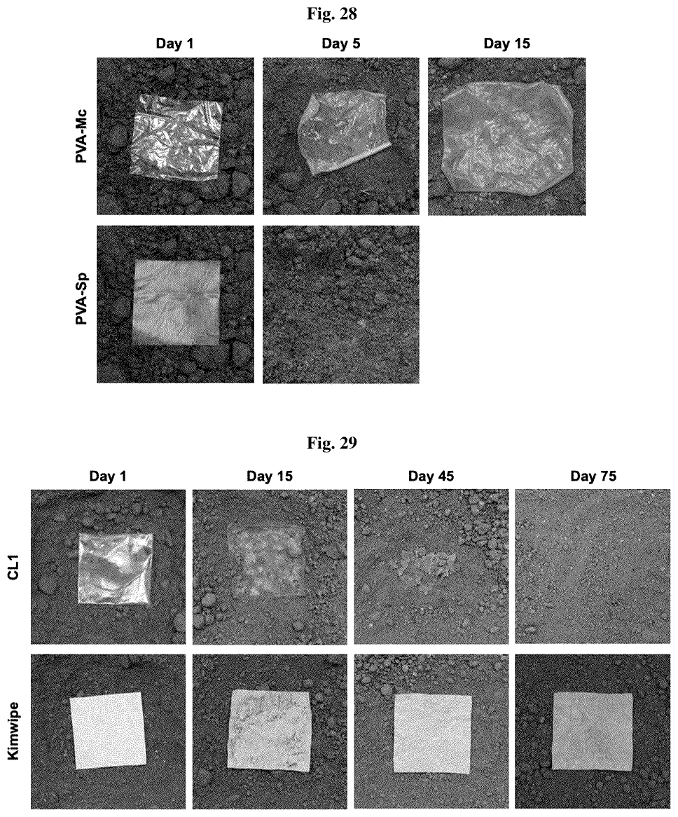

To test the relative compostability of MECHS films compared to other conventional plastics and bioplastics, we buried samples of each in a commercially available compost called fishnure, derived from fish manure. Experiments were performed in a mini greenhouse setup with samples of uniform size and shape (FIGS. 25 and 26). Under these conditions, MECHS films biodegraded completely in 15 days, while all the other samples did not (FIGS. 4A, 4C and FIGS. 26-28). Toilet paper and kimwipes biodegraded to 70% and 40%, respectively (FIGS. 4A, 4C and FIG. 26). The bioplastics, cellulose acetate (CA) and poly-L-lactic acid (PLLA) were biodegraded by 13% and 1% respectively, whereas the petrochemical plastics polyethylene terephthalate (PET) and low-density polyethylene (LDPE) did not show any biodegradation (FIG. 27). On the other hand, two different commercial polyvinyl alcohol (PVA) formulations, PVA-Mc and PVA-Sp, lost 17% weight and completely disappeared in 5 days, respectively (FIG. 28).

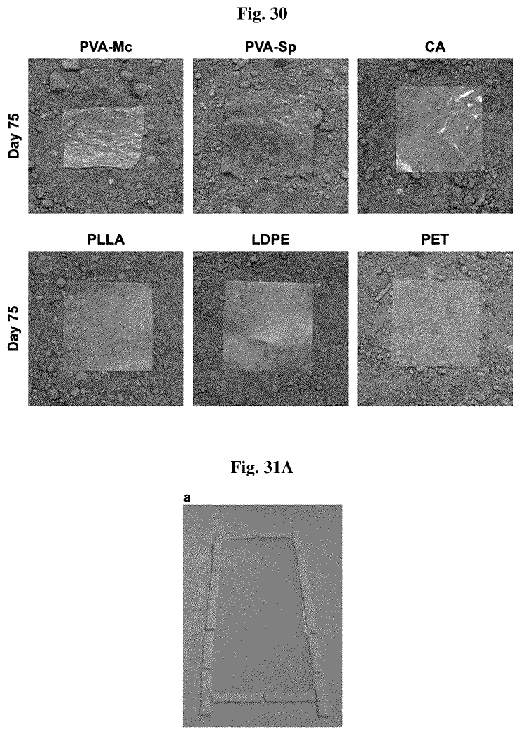

Some of the mass loss in the experiments above may have been attributable to dissolution in the moist fresh fishnure, rather than biodegradation, especially for MECHS and PVA. Therefore, we performed additional compostability tests in fishnure that was dried (i.e., placed in the greenhouse for 50 days). Under these conditions, MECHS films were able to biodegrade completely in 75 days (FIGS. 4B, 4C and Supplementary FIG. 29). The toilet paper, kimwipe and CA were found to degrade by about 60%, 16% and 13%, respectively, whereas PLLA, PET and LDPE did not show any biodegradation in dry fishnure (FIGS. 4B, 4C, FIGS. 29 and 30). However, PVA-Mc had nearly 10% weight loss, whereas PVA-Sp was found to be intact even after 75 days in dry fishnure. We could not determine the weight loss of PVA-Sp as the film was firmly sticking to the fishnure granules. These experiments show that the MECHS films are completely compostable and that their biodegradation compares favorably to many plastics, bioplastics and even toilet paper.

For potential use of MECHS as flushable packaging materials, we tested its ability to dissolve in water (FIGS. 4D, 4E). The MECHS films did not dissolve completely, likely due to the network of hydrophobic Curli nanofibers. We speculate that the more water-soluble components like glycerol, SDS and the other cellular biomass leach into water more readily. PVA-Sp dissolves completely in water whereas PVA-Mc dissolves only partially, leaving behind water-insoluble strips possibly compromising its biodegradation (FIGS. 4F, 4G). Furthermore, except for MECHS, all the other plastics compared here are composed only of carbon, hydrogen, and oxygen. Therefore, their biodegradation is often considered, in terms of breakdown completely, to lead to carbon dioxide. However, MECHS is largely composed of protein, making it the only plastic amongst those compared here with any significant nitrogen content. Therefore, it may be reasonable to consider its potential as a biofertilizer to support plant growth (FIG. 4H). Further, MECHS films could also be healed and welded by using microliters of water at the site of abrasion or attachment and subsequently ambient dried (FIGS. 4I, 4J).

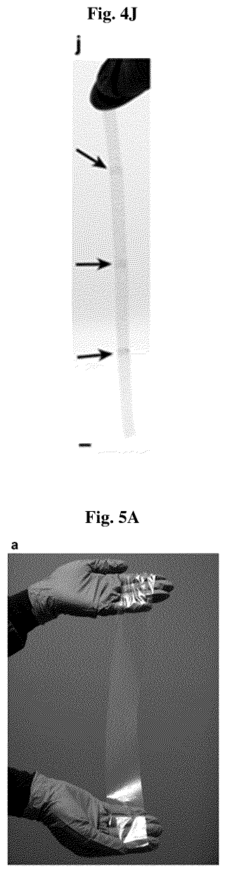

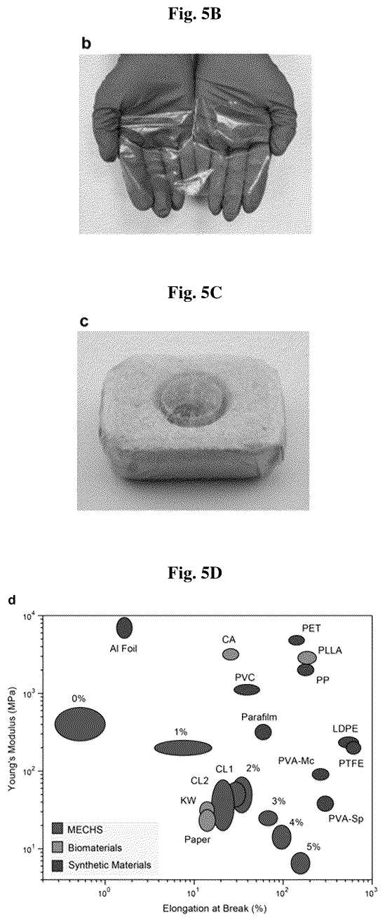

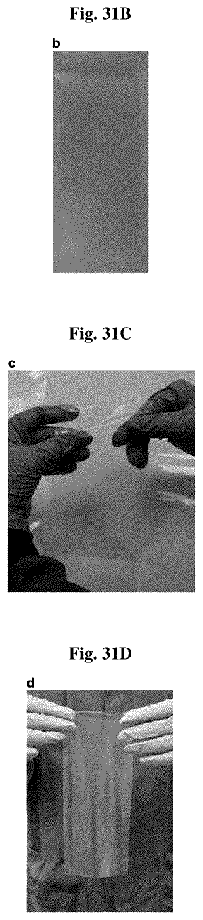

The fabrication method presented in this work yielded 500 to 1000 mg of MECHS films per liter of culture, which is nearly 10 times higher than the 50 to 100 mg obtained from our previously reported AquaPlastic protocol. In addition, the MECHS biofabrication method speeds up the process in comparison to the tedious and slow filtration process utilized for AquaPlastic. We achieved these yields even with a standard shake-flask format that is routinely used in laboratory settings for recombinant protein production. Therefore, tens of liters of bacterial culture could be used to fabricate large MECHS prototypes, such as thin films, tens of centimeters in one dimension (FIGS. 5A, 5B and FIG. 31). We also created a detergent pod as an example of flushable and biodegradable primary package (FIG. 5C).

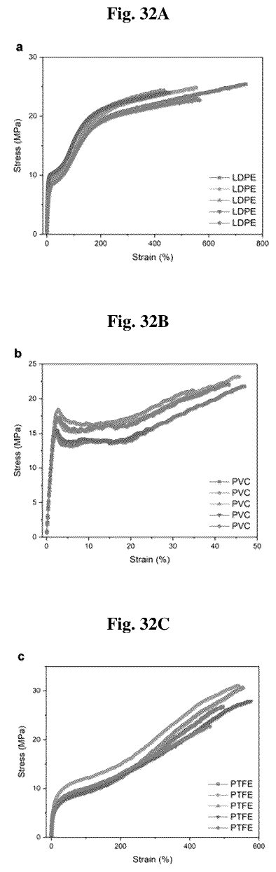

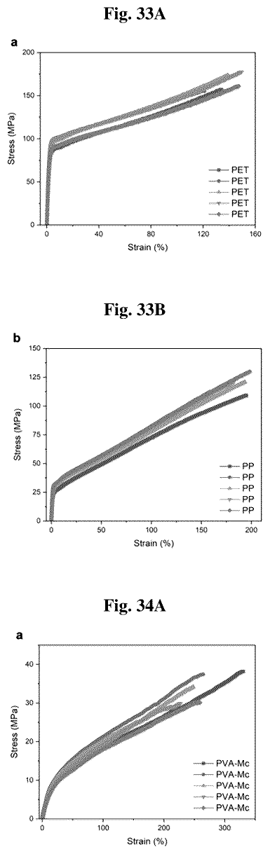

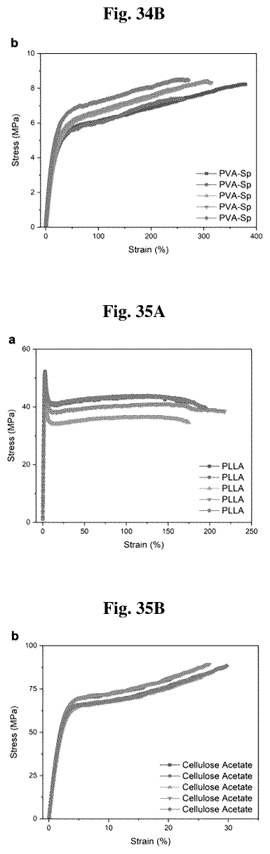

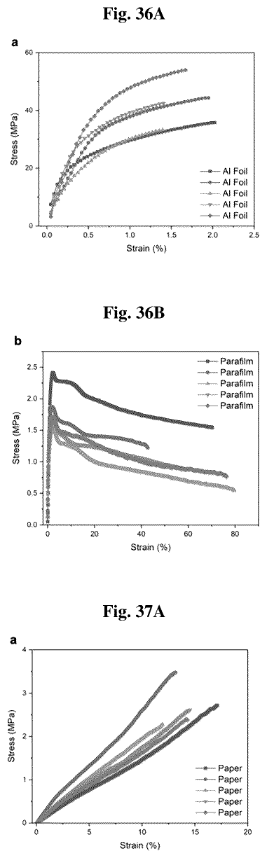

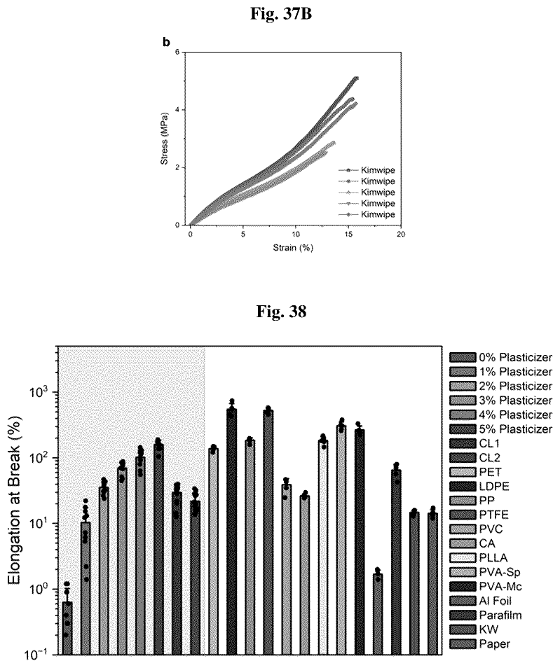

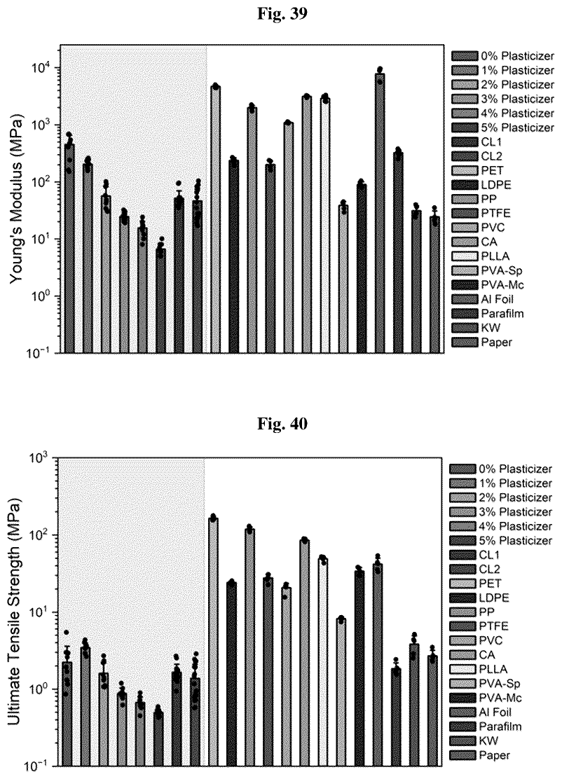

To better visualize and compare the mechanical properties of MECHS, we present an Ashby plot of Young's modulus and elongation at break for various plastics, bioplastics, biomaterials, and synthetic materials (FIG. 5D, FIG. 32-40). It is thus evident that the Young's modulus of MECHS is in the same range of LDPE, PTFE (polytetrafluoroethylene), PVA, and paper, while its elongation at break matches that of CA, PVC (polyvinyl chloride), PP (polypropylene), PET, PLLA and parafilm.

### INCORPORATION BY REFERENCE

Each of the patents, published patent applications, and non-patent references cited herein are hereby incorporated by reference in their entirety.

### EQUIVALENTS

Those skilled in the art will recognize, or be able to ascertain using no more than routine experimentation, many equivalents to the specific embodiments described herein. Such equivalents are intended to be encompassed by the following claims.

1. An engineered fiber, comprising a first Curli protein, and a second Curli protein.

2. The engineered fiber of claim 1, wherein the first Curli protein is fused to a first binding peptide, and the second Curli protein is fused to a second binding peptide.

3. The engineered fiber of claim 2, wherein the first binding peptide is linked to the second binding peptide.

4. The engineered fiber of claim 3, wherein the first binding peptide is covalently linked to the second binding peptide.

5. The engineered fiber of claim 1, wherein the first Curli protein and the second Curli protein are CsgA.

6. The engineered fiber of claim 5, wherein the CsgA comprises the amino acid sequence set forth in SEQ ID NO: 1.

7. The engineered fiber of claim 2, wherein the first binding peptide and the second binding peptide are any one of the following pairs: SpyCatcher and Spytag, SnoopCatcher and Snooptag, and streptavidin and biotin.

8. The engineered fiber of claim 7, wherein the first binding peptide and the second binding peptide are SpyCatcher and Spytag; and the Spytag comprises the amino acid sequence set forth in SEQ ID NO: 3 and the SpyCatcher comprises the amino acid sequence set forth in SEQ ID NO: 4.

9. The engineered fiber of claim 2, wherein the first Curli protein is fused to the first binding peptide via a spacer and/or linker.

10. The engineered fiber of claim 2, wherein the second Curli protein is fused to the second binding peptide via a linker and/or a spacer.

11. The engineered fiber of claim 9, wherein the linker is glycine serine linker.

12. The engineered fiber of claim 11, wherein the glycine serine linker comprises the amino acid sequence set forth in any one of SEQ ID NOs: 2, 11, and 13-18 and the sequence GSG.

13. The engineered fiber of claim 9, wherein the spacer comprises an intrinsically disordered protein.

14. The engineered fiber of claim 13, wherein the intrinsically disordered protein is TDP-43, FUS, SMN, ataxin-2, optineurin, angiogenin, tau, α-synuclein, huntingtin, DRPLA protein, elastin, or casein.

15. The engineered fiber of claim 14, wherein the casein comprises the amino acid sequence set forth in SEQ ID NO: 5.

16. The engineered fiber of claim 1, wherein the fiber is a nonfiber (e.g., a diameter of 1-100 nanometers).

17. The engineered fiber of claim 1, wherein the engineered fiber has a Young's modulus of 6-450 MPa.

18. (canceled)

19. An engineered microbial cell comprising an exogenous nucleic acid encoding a first Curli protein and a second Curli protein.

20.-36. (canceled)

37. An engineered living material, comprising a plurality of the engineered microbial cells of claim 19; and an engineered fiber comprising a first Curli protein, and a second Curli protein.

38.-40. (canceled)

41. A method of fabricating an engineered living material, comprising proliferating a plurality of engineered microbial cells of claim 19, thereby producing an engineered fiber comprising a first Curli protein, and a second Curli protein and engineered microbial cells; isolating the engineered microbial cells and engineered fiber; and allowing the isolated cells and engineered fiber to dry; thereby forming the engineered living material.

42.-46. (canceled)


SEQUENCE LISTING (19 sequences)

SEQ ID NO: 1 - protein, 131 aa, synthetic construct
GVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 2 - protein, 36 aa, synthetic construct
GGSGSSGSGGSGGGSGSSGSGGSGGGSGSSGSGGSG

SEQ ID NO: 3 - protein, 16 aa, synthetic construct
RGVPHIVMVDAYKRYK

SEQ ID NO: 4 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 5 - protein, 225 aa, synthetic construct
KVLILACLVALALARETIESLSSSEESITEYKQKVEKVKHEDQQQGEDEHQDKIYPSFQPQPLIYPFVEPIPYGFLPQNILPLAQPAVVLPVPQPEIMEVPKAKDTVYTKGRVMPVLKSPTIPFFDPQIPKLTDLENLHLPLPLLQPLMQQVPQPIPQTLALPPQPLWSVPQPKVLPIPQQVVPYPQRAVPVQALLLNQELLLNPTHQIYPVTQPLAPVHNPISV

SEQ ID NO: 6 - DNA, 393 bp, synthetic construct
  source 1..393: mol_type = other DNA
ggtgttgttcctcagtacggcggcggcggtaaccacggtggtggcggtaataatagcggcccaaattctgagctgaacatttaccagtacggtggcggtaactctgcacttgctctgcaaactgatgcccgtaactctgacttgactattacccagcatggcggcggtaatggtgcagatgttggtcagggctcagatgacagctcaatcgatctgacccaacgtggcttcggtaacagcgctactcttgatcagtggaacggcaaaaattctgaaatgacggttaaacagttcggtggtggcaacggtgctgcagttgaccagactgcatctaactcctccgtcaacgtgactcaggttggctttggtaacaacgcgaccgctcatcagtac

SEQ ID NO: 7 - DNA, 108 bp, synthetic construct
  source 1..108: mol_type = other DNA
ggtggatctggtagcagcggctctggtggttctgggggcggaagtggctcctctgggagcggggggtcgggtggtggctcgggttcatctggtagtggcggttcgggt

SEQ ID NO: 8 - DNA, 48 bp, synthetic construct
  source 1..48: mol_type = other DNA
cgcggcgtgccgcatattgtgatggtggatgcgtataaacgctataaa

SEQ ID NO: 9 - DNA, 339 bp, synthetic construct
  source 1..339: mol_type = other DNA
gtgaccaccctgagcggcctgagcggcgaacagggcccgagcggcgatatgaccaccgaagaagatagcgcgacccatattaaatttagcaaacgcgatgaagatggccgcgaactggcgggcgcgaccatggaactgcgcgatagcagcggcaaaaccattagcacctggattagcgatggccatgtgaaagatttttatctgtatccgggcaaatatacctttgtggaaaccgcggcgccggatggctatgaagtggcgaccccgattgaatttaccgtgaacgaagatggccaggtgaccgtggatggcgaagcgaccgaaggcgatgcgcatacc

SEQ ID NO: 10 - DNA, 678 bp, synthetic construct
  source 1..678: mol_type = other DNA
atgaaagtgctgattctggcgtgcctggtggcgctggcgctggcgcgcgaaaccattgaaagcctgagcagcagcgaagaaagcattaccgaatataaacagaaagtggaaaaagtgaaacatgaagatcagcagcagggcgaagatgaacatcaggataaaatttatccgagctttcagccgcagccgctgatttatccgtttgtggaaccgattccgtatggctttctgccgcagaacattctgccgctggcgcagccggcggtggtgctgccggtgccgcagccggaaattatggaagtgccgaaagcgaaagataccgtgtataccaaaggccgcgtgatgccggtgctgaaaagcccgaccattccgttttttgatccgcagattccgaaactgaccgatctggaaaacctgcatctgccgctgccgctgctgcagccgctgatgcagcaggtgccgcagccgattccgcagaccctggcgctgccgccgcagccgctgtggagcgtgccgcagccgaaagtgctgccgattccgcagcaggtggtgccgtatccgcagcgcgcggtgccggtgcaggcgctgctgctgaaccaggaactgctgctgaacccgacccatcagatttatccggtgacccagccgctggcgccggtgcataacccgattagcgtg

SEQ ID NO: 11 - protein, 15 aa, synthetic construct
GGGGSGGGGSGGGGS

SEQ ID NO: 12 - skipped

SEQ ID NO: 13 - protein, 4 aa, synthetic construct
GSGG

SEQ ID NO: 14 - protein, 5 aa, synthetic construct
GGSGG

SEQ ID NO: 15 - protein, 10 aa, synthetic construct
GGSGGGGSGG

SEQ ID NO: 16 - protein, 15 aa, synthetic construct
GGSGGGGSGGGGSGG

SEQ ID NO: 17 - protein, 6 aa, synthetic construct
SGGSGG

SEQ ID NO: 18 - protein, 10 aa, synthetic construct
GGGGSGGGGS

SEQ ID NO: 19 - protein, 18 aa, unidentified
  source 1..18: note = amyloid core domain sequence
SXXXXXQXGXGNXAXXXQ